(19) United States
(12) Patent Application Publication
(10) Pub. No.: US 2021/0373102 A1
(43) Pub. Date: Dec. 2, 2021

(54) ORIENTATION-INDEPENDENT ORDER PARAMETER DERIVED FROM MAGNETIC RESONANCE R1P DISPERSION IN ORDERED TISSUE

(71) Applicant: THE REGENTS OF THE UNIVERSITY OF MICHIGAN, Ann Arbor, MI (US)

(72) Inventor: Yuxi Pang, Superior Township, MI (US)

(21) Appl. No.: 17/313,591

(22) Filed: May 6, 2021

Related U.S. Application Data
(60) Provisional application No. 63/022,155, filed on May 8, 2020.

(51) Int. Cl. G01R 33/50; A61B 5/055; A61B 5/00

(52) U.S. Cl. CPC G01R 33/50; A61B 5/055; A61B 5/4523; A61B 5/4519; A61B 5/4514

(57) ABSTRACT

Techniques for analyzing ordered tissue to calculate an orientation-independent order parameter S that is sensitive to the collagen microstructural integrity in cartilage are provided. An magnetic resonance image of ordered tissue may be acquired, and based on the image, an R_{1ρ} dispersion of the ordered tissue may be measured. R₂^{a}(α) and τ_{b}(α) values for the ordered tissue may be derived based on the measured R_{1ρ} dispersion of the ordered tissue. An orientation-independent order parameter S may be calculated for the ordered tissue using the following equation:

$$S = \sqrt{\frac{2}{3 {d}^{2}} \frac{{R}_{2}^{a} ( α )}{{τ}_{b} ( α )}} .$$

The level of degeneration of the ordered tissue may be determined based on the orientation-independent order parameter S for the ordered tissue. In order to derive this valuable order parameter efficiently and reliably in clinical studies, an optimized spin-lock preparation strategy was introduced, including a novel fully-refocused spin-locking pulse sequence and a constant R_{1ρ} weighting with both spin-lock duration and strength being altered simultaneously.

### CROSS REFERENCE TO RELATED APPLICATIONS

The present application claims priority to U.S. Provisional Patent Application No. 63/022,155, filed May 8, 2020, entitled “An Orientation-Independent Order Parameter Derived from Magnetic Resonance R_{1ρ} Dispersion Imaging in Ordered Tissue,” the disclosure of which is incorporated herein by reference in its entirety.

### STATEMENT OF GOVERNMENT INTEREST

This invention was made with government support under R01HD093626 awarded by the National Institutes of Health. The government has certain rights in the invention.

### FIELD OF THE DISCLOSURE

The present disclosure generally relates to a method of determining (i.e. measuring and calculating) the ordered water in biological tissues to reveal their specific constituents' microstructural integrities such as in articular cartilage with degenerated collagen.

### BACKGROUND

The background description provided herein is for the purpose of generally presenting the context of the disclosure. Work of the presently named inventor, to the extent it is described in the background section, as well as aspects of the description that may not otherwise qualify as prior art at the time of filing, are neither expressly nor impliedly admitted as prior art against the present disclosure.

Magnetic resonance R₂ imaging of ordered tissue exhibits a well-known magic angle effect that tends to overshadow pathological changes in the ordered tissue. Consequently, it is challenging to reliably diagnose early degeneration of ordered tissue (e.g., such as cartilage) in clinical practice.

Generally speaking, water is ubiquitous and it is not as uniform as it appears in living systems. Many highly structured (i.e., highly ordered) tissues can be found in the human body, including peripheral nerves, white matter, skeletal and myocardial muscles, tendons and articular cartilage. Magnetic resonance (MR) imaging of these specialized tissues exhibits a well-known orientation-dependent phenomenon, referred to as magic angle effect, predominantly in transverse R₂ relaxation measurements. In the last two decades, the compositional MR imaging has received great attention in characterizing early cartilage degeneration secondary to osteoarthritis (OA), a common joint disease affecting mostly an aging population and young athletes after surgical treatments on anterior cruciate ligament (ACL) injuries.

One of the hallmark features of OA is a progressive loss of cartilage and no disease-modifying drug is available to date. Hence, it is especially critical to have an effective noninvasive imaging means to detect early cartilage degradation in order to prevent further adverse OA progression with potentially new therapeutic interventions or simply regular diet modifications. To this end, a number of advanced MR imaging techniques have been developed, two of which in particular have been investigated extensively in clinical studies, i.e. water proton R₂ (1/T₂) and longitudinal R_{1ρ} (1/T_{1ρ}) relaxation in a rotating frame.

To date, the biophysical mechanisms underlying R₂ and R_{1ρ} relaxations, induced by water and structural protein interactions on relatively slow time scales, had been controversial despite being widely used and a growing body of clinical evidence is shedding light on which structural protein has been probed. More than 50 years ago, Berendsen discovered that water bound to collagen triple-helix secondary structures give rise to an orientation-dependent MR resonance doublet splitting and then proposed that bound water form a chainlike structure along collagen fibers in hydrated cartilage. This orientation-dependent MR phenomenon was later rediscovered by Fullerton et al. in clinical MR imaging of tendon and then investigated in-depth by others with high-field and high-resolution microcopy MR imaging techniques on various cartilage samples, some of which were enzymatically degraded to deplete a specific structural protein such as collagen (CA) or proteoglycan (PG). The reported relaxation measurements from these CA− and/or PG− depleted samples revealed that the water-CA interactions in terms of residual dipolar coupling (RDC) is the dominant relaxation mechanism in clinical R₂ and R_{1ρ} studies in which the static magnetic fields B₀ are usually less than or equal to 3T.

It was not without any contention regarding this dominant relaxation mechanism, and the chemical exchange (CHEX) effect in terms of water-PG interactions was also considered, and great effort has been made to enhance clinical imaging data acquisitions and standardize the pulse sequences across different imaging systems. However, no convincing clinical evidence has yet been demonstrated to corroborate the proposed mechanism. On the contrary, many clinical and experimental studies have provided substantial data to substantiate RDC as the prevailing relaxation mechanism. For instance, Xia et al. showed that the measured R₂ at 7T on canine cartilage specimens decreased about 10-20% after PG depletion when the samples were orientated at the magic angle (i.e. RDC=0). Had these measurements been carried out at 3T instead of 7T, the reported decreases in R₂ due to PG− depletion would have been reduced to a few percent, implying that the CHEX effect will not significantly contribute to R₂ and R_{1ρ} at 3T.

Retrospectively, it was Mlynarik et al. who provided indisputable evidence to unravel the above-mentioned controversy. He concluded that R₂ and R_{1ρ} in clinical studies (B₀≤3T) were mainly induced by RDC resulting from the slow anisotropic motion of water molecules restricted in the collagen matrix. In a recent comprehensive study of relaxation anisotropy on bovine patellar cartilage samples at 9.4T, a very large number of MR relaxation metrics had been investigated in-depth and anisotropic R₂ was found to be the most sensitive metric to cartilage degenerative alterations. In order to effectively and efficiently extract this potential relaxation parameter, a novel approach based on a single T2W sagittal image, referred to as anisotropic R₂ of collagen degeneration (ARCADE), was proposed as an alternative to a time-consuming and much involved composite relaxation metric R₂-R_{1ρ}, which had been demonstrated to measure only an incomplete anisotropic R₂ in clinical studies.

Although good progress has been made so far in measuring anisotropic R₂ in standard clinical studies without significantly lengthening scanning time, it is still challenging to make the reliable diagnosis of early cartilage degeneration because of the well-known magic angle effect. This grave situation has been clearly highlighted in a recent study, showing that the changes in R₂ and R_{1ρ} values due to the magic angle effect could be several times more than that caused by cartilage degeneration. As a result, the potential of the compositional MR imaging as a biomarker for cartilage degeneration has been compromised particularly for the diagnostic purpose. Therefore, it is crucial to develop a novel method to overcome the magic angle effect and yet to retain the intrinsic sensitivity of anisotropic R₂. Currently, a few initial attempts have been made to uncouple the magic angle effect; unfortunately, the most important sensitivity to the underlying microstructural changes was also lost in those proposed methods by either lengthening echo-time (TE) or utilizing T1 relaxation (e.g. in MT sequence) that is not specific to any involved constituents in cartilage extracellular matrix (ECM).

Water proton magnetic relaxation is not only one principal factor governing an exquisite and diverse soft-tissue contrast in clinical MR imaging, but also one powerful tool for studying in detail the structural and dynamical information about water molecules in various biological systems. In this regard, the field-dependent longitudinal relaxation R_{1ρ} dispersion in a rotating frame has been revealed to provide a unique insight into water-macromolecule interactions. To some extent, R_{1ρ} can be viewed as transverse relaxation R₂ under the influence of a spin-lock (SL) RF pulse, and it is sensitive exclusively to low-frequency water molecular interactions. As early as 1970s, R_{1ρ} had been used to investigate pathophysiological changes in biological tissues. About 20 years later, the first R_{1ρ} imaging study of articular cartilage to characterize osteoarthritis (OA) was reported, and since then, considerable efforts have been made to develop and standardize R_{1ρ} mapping methodology across primary MR scanner platforms in clinical environments.

R_{1ρ} mapping of articular cartilage has been motivated by the diagnostic and research-based utility of a noninvasive and sensitive imaging method, which could detect early cartilage degeneration in the absence of advanced macroscopic changes apparent on standard anatomical MR imaging. When R_{1ρ} was first proposed as a promising MR biomarker for characterizing changes in proteoglycan (PG) content—a major biochemical component in articular cartilage, the specificity of R_{1ρ} changes to PG alterations was unclear and this topic has remained a point of controversy. For instance, two early studies from the 2000s did not support the concept that R_{1ρ} itself could be a sensitive biomarker of PG in OA cartilage, and, to date, a large amount of clinical data has been in agreement with the findings from these two landmark studies.

It has been suggested that R_{1ρ} dispersion rather than R_{1ρ} itself was sensitive to early cartilage degeneration, and the proposed composite relaxation metric R₂-R_{1ρ} has substantiated this concept. As previously shown, R₂-R_{1ρ} is merely a two-point R_{1ρ} dispersion in which R₂ is basically an R_{1ρ} acquired with the SL RF strength ω₁/2π=0, and R_{1ρ} is normally measured with ω₁/2π=500 Hz. Most importantly, a theoretical framework of R_{1ρ} dispersion has been outlined for highly structurally-ordered tissues such as articular cartilage, and the observed R_{1ρ} dispersion can be associated directly with those water molecules contained within the triple-helix interstices from collagen microstructure. Thus, R_{1ρ} dispersion can be potentially exploited as a specific MR biomarker to detect early collagen degeneration in joint OA or collagen accumulation in some tissue fibrosis.

In order to utilize R_{1ρ} dispersion imaging in clinical studies, a reliable acquisition protocol that does not significantly lengthen imaging time is required. Currently, the developed 3D MAPSS sequence can be considered as the state-of-the-art R_{1ρ} mapping of knee cartilage, and it is being promoted as a standard across different MR scanners. This dedicated R_{1ρ} mapping strategy was established from the widely used magnetization-prepared turbo-FLASH sequence in which RF phase cycling and tailored excitation angles were employed to mitigate the potential imaging artifacts. These imaging artifacts could be respectively induced during the SL preparation by non-uniform B₀ and B₁ fields, and during imaging readout by transient magnetization evolution towards steady-state (i.e. T₁ relaxation effect).

Although R_{1ρ} can be accurately quantified with 3D MAPSS, the scan time is doubled when compared with a standard albeit inaccurate R_{1ρ} mapping with no RF phase cycling. Furthermore, this advanced 3D MAPSS sequence was initially designed for R_{1ρ} mapping (i.e. with one ω₁/2π) but not for R_{1ρ} dispersion (i.e. with multiple ω₁/2π). Thus, it is unclear to what extent the prepared SL magnetization will be compromised by 3D MAPSS particularly when ω₁/2π becomes relatively small.

The various SL schemes reported in the literature have not been tailored to R_{1ρ} dispersion but rather optimized for some specific R_{1ρ} mapping scenarios using an extreme ω₁/2π at higher B₀ fields.

### SUMMARY

In one embodiment, a computer-implemented method is provided. The computer-implemented method comprises: acquiring, by a processor, a magnetic resonance image of an ordered tissue; measuring, by a processor, based on the magnetic resonance image of the ordered tissue, an R_{1ρ} dispersion of the ordered tissue; deriving, by a processor, R₂^{a}(α) and τ_{b}(α) for the ordered tissue based on the measured R_{1ρ} dispersion of the ordered tissue; calculating, by a processor, an orientation-independent order parameter S for the ordered tissue, using the following equation:

$$S = \sqrt{\frac{2}{3 {d}^{2}} \frac{{R}_{2}^{a} ( α )}{{τ}_{b} ( α )}} ;$$

and determining, by a processor, based on the orientation-independent order parameter S for the ordered tissue, a level of degeneration of the ordered tissue.

In another embodiment, a system is provided. The system comprises a magnetic resonance imaging (MRI) device configured to capture a magnetic resonance image of an ordered tissue; one or more processors; and one or more memories storing instructions. The instructions, when executed by the one or more processors, cause the one or more processors to: measure, based on the magnetic resonance image of the ordered tissue, an R_{1ρ} dispersion of the ordered tissue; derive R₂^{a}(α) and τ_{b}(α) for the ordered tissue based on the measured R_{1ρ} dispersion of the ordered tissue; calculate an orientation-independent order parameter S for the ordered tissue, using the following equation:

$$S = \sqrt{\frac{2}{3 {d}^{2}} \frac{{R}_{2}^{a} ( α )}{{τ}_{b} ( α )}} ;$$

and determine, based on the orientation-independent order parameter S for the ordered tissue, a level of degeneration of the ordered tissue.

In still another embodiment, a tangible, non-transitory computer-readable medium is provided. The tangible, non-transitory computer-readable medium stores executable instructions that, when executed by at least one processor of a computing device, cause the computing device to: acquire a magnetic resonance image of an ordered tissue; measure, based on the magnetic resonance image of the ordered tissue, an R_{1ρ} dispersion of the ordered tissue; derive R₂^{a}(α) and τ_{b}(α) for the ordered tissue based on the measured R_{1ρ} dispersion of the ordered tissue; calculate an orientation-independent order parameter S for the ordered tissue, using the following equation:

$$S = \sqrt{\frac{2}{3 {d}^{2}} \frac{{R}_{2}^{a} ( α )}{{τ}_{b} ( α )}} ;$$

and determine, based on the orientation-independent order parameter S for the ordered tissue, a level of degeneration of the ordered tissue.

### BRIEF DESCRIPTION OF THE DRAWINGS

Table 1 illustrates partitioned transverse relaxation R₂ absolute (1/s) and relative (%) rates, average orientation-dependent R_{1ρ} dispersion parameters [symbol] τ_{b} [symbol] (μs) and [symbol] R₂^{a}(θ) [symbol] (1/s), and derived order parameters S (10⁻³) in the deep zone from four bovine patellar cartilage specimens at 9.4T. Note, θ_{MA}(°) and τₑₓ (μs) represent respectively an orientation with a minimal R₂ and a chemical exchange correlation time. All data are reported as mean±standard deviation.

Table 2 illustrates average measured and modeled R_{1ρ} dispersion parameters in the femoral, tibial and patellar cartilage from one live human knee. All data are reported as mean±standard deviation.

Table 3 illustrates tailored spin-lock RF durations (“spin-lock time” or “TSL”) and strengths or powers (PWR, i.e. ω₁/2π) for the constant magnetization preparations (Mₚᵣₑₚ) used in quantitative R_{1ρ} dispersion imaging protocol. Note that these specific values were determined assuming R₂ⁱ=R₂^{a}20 (1/s) and τ_{b}=300 (μs).

Table 4 illustrates simulated noisy R_{1ρ} dispersion quantification under influences of various SNR, with (+) and without (−) an internal reference. The key input model parameters were given as follows: R₂ⁱ=R₂^{a}=20 (1/s) and =τ_{b}=300 (μs), and simulations were performed for different prepared R_{1ρ} magnetization (Mₚᵣₑₚ). The group of “All” includes all three Mₚᵣₑₚ groups, i.e. 50%+60%+70%. Note that an order parameter S (10⁻³) of 2.052 can be determined herein given the values of R₂^{a} and τ_{b}.

Table 5 illustrates quantitative dispersion with (+) and without (−) an internal reference (REF1) for two radially-segmented ROIs (i.e. SZ and DZ of the tibial cartilage) from the first subject's left knee. Note that the “All” group includes all three Mₚᵣₑₚ groups, i.e. 50%+60%+70%, and the fitting results for DZ are displayed in FIG. 14. In Table 5, “DZ” means deep zone; “ROI” means region of interest; and “SZ” means superficial zone.

Table 6 illustrates quantitative dispersion (=60%) of all knees (n=6), with the second subject (i.e. S2L01 and S2L02) and the third subject having their left knees re-scanned 3 months later. In Table 6, “L” means left; “R” means right; and “S” means subject.

Table 7 illustrates repeated synthetic and measured (=500 Hz) for the second and third subjects. In Table 7, “DZ” means deep zone; “Exp” means experimental or measured; “Syn” means synthetic; and “SZ” means superficial zone.

FIG. 1 illustrates a representative (red) dipolar inter-nuclear vector H—H and an effective (black) vector <H—H> alignment in a triple-helix model peptide (A), according to a molecular dynamics simulation study (Copyright© 2016, American Chemical Society). The <H—H> vector (i.e. OA) is characterized without (B) and with (C) an axially symmetric model with its rotational axis in red (7i).

FIG. 2 illustrates orientation-dependent depth-profile maps for T₂ (A) and standard T_{1ρ} relaxation times (ms) with a spin-lock RF strength (ω₁/2π) of 2000 Hz (B) from one bovine cartilage sample (B1S2). A horizontal axis starts from articular surface (0%) to bone interface (100%) and the deep zone is defined between 40% and 80% in depth indicated by two vertical dashed lines (B).

FIG. 3 illustrates orientation-dependent R_{1ρ} (1/s) relaxations with ω₁/2π=0 (red), 0.25 (green) and 2 kHz (blue) for the same sample B1S2 (A), with the solid lines standing for the best fits to R₂^{a}(3 cos² θ−1)²/4 for the averages in the deep zone indicated by the dashed lines in the middle of shaded areas (±standard deviations), and two R_{1ρ} (1/s) dispersions when the sample orientated at θ=20° (B) and 60° (C.) relative to B₀.

FIG. 4 illustrates a scatterplot (A) of τ_{b} (μs) and R₂^{a}(θ) (1/s) and a box-and-whisker diagram (B) of the derived order parameter S (10⁻³) for each of four bovine patellar samples. The order parameter S was only calculated when samples orientated <50° (B1S1 and B₂S3) or <35° (B1S2 and B₁S3) to avoid potential diminishing R₂^{a}(θ) near the magic angles.

FIG. 5 illustrates three representative segmented ROIs highlighted by colored arrows in the femoral (red), tibial (green) and patellar (blue) cartilage on one R_{1ρ}-weighted (ω₁/2π=125 Hz, TSL=1 ms) sagittal image slice (A) and the corresponding R₁-, dispersion curve fittings (solid lines) in the deep zone (B), with error bars standing for the measured R_{1ρ} standard deviations.

FIG. 6 illustrates R_{1ρ} relative distributions (%) in the femoral (red solid), tibial (green dot-dash) and patellar (blue dash) cartilage from one live human knee, dispersed with ω₁/2π=125 Hz (A), 500 Hz (B) and 1000 Hz (C) and the fitted model parameters of R₂ⁱ (D), τ_{b} (E) and R₂^{a}(θ) (F) for the measured R_{1ρ} dispersions.

FIG. 7 illustrates a scatterplot (A) of τ_{b} (μs) and R₂^{a}(θ) (1/s) and a box-and-whisker diagram (B) of the derived order parameter S (10⁻³) from the femoral (red circles), tibial (green squares) and patellar (blue triangles) cartilage in one live human knee.

FIG. 8 illustrates order parameter S comparisons among human and bovine normal cartilages (A), between two grades of osteoarthritis (OA) in human knee tibial cartilage samples (B) and among enzymatically modified bovine patellar cartilage samples (C).

FIGS. 9A-9E illustrate two key components in an optimized SL prepared turbo-FLASH sequence (FIGS. 9A-9B), a representative (normalized) R_{1ρ}-weighting map (FIG. 9C), two examples of prepared transient magnetization towards steady-state evolutions (FIG. 9D) and a k-space filling pattern in two phase-encoding directions (FIG. 9E). Note that TSL and ω₁/2π were respectively limited to [9, 32] (ms) and [0, 1000] (Hz), T_{seg}=2000 (ms), constant excitation FA α₀=13°, TR=6.8 (ms) and N=64. In FIGS. 9A-9E, “FA” means flip angle; “FLASH” means fast low angle shot; “ms” means millisecond; “SL” means spin-lock; and “TSL” means spin-lock time.

FIGS. 10A-10F illustrate simulated noisy R_{1ρ} dispersion quantification with (+, solid line) and without (−, dashed line) an internal reference (REF) under various SNR conditions for different R_{1ρ}-weighting preparations: Mp=50% (blue), 70% (red) and All (i.e. 50%+60%+70%, black). The quantification accuracy indicated by RMSE (%) is shown respectively for R2i, R2a and τb in FIGS. 10A-10C, and the fitted precision for R2i is presented in FIG. 10D. The fitting biases (%) due to a relative uncertainty δR₂ⁱ (%) are displayed in FIG. 10E for R₂ⁱ (black), R₂^{a} (red), τ_{b} (green) and S (blue), and an example of such a biased fitting (black solid line) is demonstrated in FIG. 10F. In FIGS. 10A-10F, “Mp” means magnetization preparation; “RMSE” means root mean square error; and “SNR” means signal-to-noise ratio.

FIGS. 11A-11D illustrate optimal excitation FA (°) profiles (FIG. 11A) calculated with TR=6.8 (ms) and T₁=1240 (ms) for Mₚᵣₑₚ from 0 to 100 (%) with N=32 (red), 64 (green), 96 (blue) and 128 (black), an average magnetization obtained from the prepared Mₚᵣₑₚ using various FAs and N=64 (FIG. 11B), signal profiles measured from femoral condyle (red), tibial (green) and patellar (blue) cartilage with FA α₀ varied from 9° to 17° (FIG. 11C) and an image slice (α₀=13°) showing ROIs from which the signal profiles were taken (FIG. 11D). In FIGS. 11A-11D, “FA” means flip angle; “Mₚᵣₑₚ” means prepared magnetization; and “ROI” means region of interest.

FIGS. 12A and 12B illustrate two R_{1ρ}-weighted (ω₁/2π=500 Hz) images acquired with the developed (improved) R_{1ρ} dispersion imaging protocol (TSL=21 ms, FIG. 12A) and the standard (original) R_{1ρ} mapping (TSL=20 ms, FIG. 12B). FIG. 12C illustrates overlaid line profiles taken at the same anatomical location from the developed (improved) R_{1ρ} dispersion imaging protocol and the standard (original) R_{1ρ} mapping. Note that the line profile (blue) from FIG. 12B was scaled up by 2, making it comparable in femoral condyle with that (red) from FIG. 12A. In FIGS. 12A and 12B, “DZ” means deep zone; “LL” means lower left, and “UR” means upper right.

FIGS. 13A-13D illustrate representative R_{1ρ} dispersion modeling (solid black lines) with an internal reference. FIG. 13A displays a sagittal imaging slice of the first subject's left knee overlaid with an angular-radial segmentation, a reference orientation (i.e. B₀ direction) and a yellow arrow pointing to an angularly-segmented ROI in the tibial cartilage. Different R_{1ρ} dispersions were presented for the SZ (FIG. 13B) and the DZ (FIG. 13C) from the segmented ROI based on all three measurements, i.e. Mₚᵣₑₚ=50% (red circle), 60% (green square), 70% (blue diamond). FIG. 13D highlights the R_{1ρ} dispersion modeling only for Mₚᵣₑₚ=60% as shown in FIG. 13C. In FIGS. 13A-13D, “DZ” means deep zone; “Mₚᵣₑₚ” means prepared magnetization; “ms” means millisecond; “P” means posterior; “REF” means internal reference; “ROI” meanas region of interest; “S” means superior; “SZ” means superficial zone; and “TSL” means spin-lock time.

FIGS. 14A-14D illustrate quantitative R_{1ρ} dispersion on all and subgroup measurements as shown in FIG. 5C, with (+, red bars) and without (−, blue bars) an REF1, for modeled R₂ⁱ (FIG. 14A), R₂^{a} (FIG. 14B), τ_{b} (FIG. 14C) and S (FIG. 14D). Note that the error bars stand for the fitting errors in terms of standard deviations. In FIGS. 14A-14D, “ROI” means region of interest; and “μs” means microsecond.

FIGS. 15A-15D illustrate two internal references (colored vertical lines) comparisons between REF1 (red) and REF2 (blue), with the former derived from MA (θ≈55°) orientations and the latter from the fitted S₀ (in logarithmic scale) distribution (FIG. 15A) and R₂ⁱ distribution (FIG. 15B) when ω₁=∞, without an REF1. The fitted R₂^{a} (FIG. 15C) and τ_{b} (FIG. 15D) histogram comparisons incorporating an REF1 (red) or an REF2 (blue) when quantifying R_{1ρ} dispersions. Note that these quantifications were performed on all the segmented ROIs in the deep femoral cartilage. In FIGS. 15A-15D, “ROI” means region of interest; and “μs” means microsecond.

FIGS. 16A-16F illustrate exemplary ROI-based parametric maps of R₂ⁱ, R₂^{a}, τ_{b}, S and R² (FIGS. 16B-16F) derived from R_{1ρ} dispersion from the third subject knee cartilage, with each superimposed on one T2W sagittal image (FIG. 16A). In FIGS. 16A-16F, “ROI” means region of interest; and “μs” means microsecond.

FIGS. 17A-17D illustrate fitted order parameter S histogram comparisons (FIGS. 17A and 17B) between two repeated scans, and similar plots (FIGS. 17C and 17D) for the synthetic (blue) and the measured (red) R1ρ (ω₁/2π=500 Hz), in the deep femoral cartilage from the second (FIGS. 17A and 17C) and the third subject (FIGS. 17B and 17C). Note that the measured R_{1ρ} was obtained using the standard R_{1ρ} mapping method, while the synthetic one was derived from the fitted R_{1ρ} dispersion model parameters. In FIGS. 17A-17D, “DZ” means deep zone.

FIGS. 18A-18D illustrate Bloch simulations for different SL performances subjected to non-uniform B₀ and B₁ field artifacts. The SL diagrams were given above the simulated z-component magnetization (M_{z}), with α_{y}, α_{-y}, and βₓ, standing respectively for flip-down, flip-up and refocusing RF pulses and 4τ for TSL. In these diagrams, RF pulse phase was indicated by x (0°), y (90°), −x (180°) and −y (270°). Note that the standard and the proposed SL schemes (discussed in this work) are shown in FIGS. 18A and 18D, and B₀ and B₁ field inhomogeneities were respectively limited, i.e. Δω₁/2π=[0, 250] (Hz) and Δω₁/2π=[0, 1000] (Hz). In FIGS. 18A-18D, “SL” means spin-lock; and “TSL” means spin-lock time.

FIGS. 19A-19D illustrate simulated R_{1ρ}-weighting (normalized) contour plots assuming τ_{b}=100, 200, 300 and 150 (μs) as shown in FIGS. 19A, 19B, 19C, and 19D, respectively. Note that all these plots were calculated using R₂ⁱ, =R₂^{a}=20 (1/s) except for that using R₂ⁱ=15 (1/s) in FIG. 19D. In FIGS. 19A-19D, “ms” means millisecond; “TSL” means spin-lock time; and “μs” means microsecond.

FIG. 20 illustrates an exemplary computer system that may be used for analysis as described here and connected to a medical imaging system.

FIG. 21 illustrates a flow diagram of an exemplary method of analyzing ordered tissue to calculate an orientation-independent order parameter S that is sensitive to the microstructural integrity of cartilage.

### DETAILED DESCRIPTION

The present disclosure provides systems and methods for analyzing ordered tissue to calculate an orientation-independent order parameter S that is sensitive to the collagen microstructural integrity in cartilage.

This orientation-dependent order parameter S may be utilized to characterize the degeneration of ordered tissue, such as cartilage, in clinical settings. A theoretical framework for developing this orientation-independent order parameter S was formulated based on R_{1ρ} dispersion coupled with an oversimplified molecular reorientation model, where anisotropic R₂ (i.e. R₂^{a}(θ)) becomes proportional to correlation time τ_{b}(θ) and an orientation-independent order parameter S can thus be established. This new methodology was corroborated on the publicly available orientation-dependent (θ=n*15°, n=0-6) R_{1ρ} dispersion (ω₁/2π=0, 0.25, 0.5. 1.0. 2.0 kHz) of bovine cartilage samples at 9.4T and R_{1ρ} dispersion (ω₁/2π=0.125, 0.25, 0.5, 0.75, 1.0 kHz) on one live human knee at 3T.

The τ_{b}(θ) derived from orientation-dependent R_{1ρ} dispersion demonstrated a significantly high correlation (r=0.89+0.05, P<0.05) with the corresponding R₂^{a}(θ) on cartilage samples, and a moderate correlation (r=0.51, P<0.01) was found in human knee. The average order parameter S (10⁻³) from bovine cartilage was almost two times larger than that from human knee, i.e. 3.90±0.89 vs. 1.80±0.05.

The order parameters derived from R_{1ρ} dispersion measurements are largely orientation-independent and thus lend strong support to the outlined theoretical framework. The promising results from this study could have great clinical implications in expanding the compositional MR imaging beyond its current applications.

The present disclosure further provides an efficient and robust R_{1ρ} dispersion mapping of human knee cartilage using tailored spin-locking in an optimized 3D turbo-FLASH sequence.

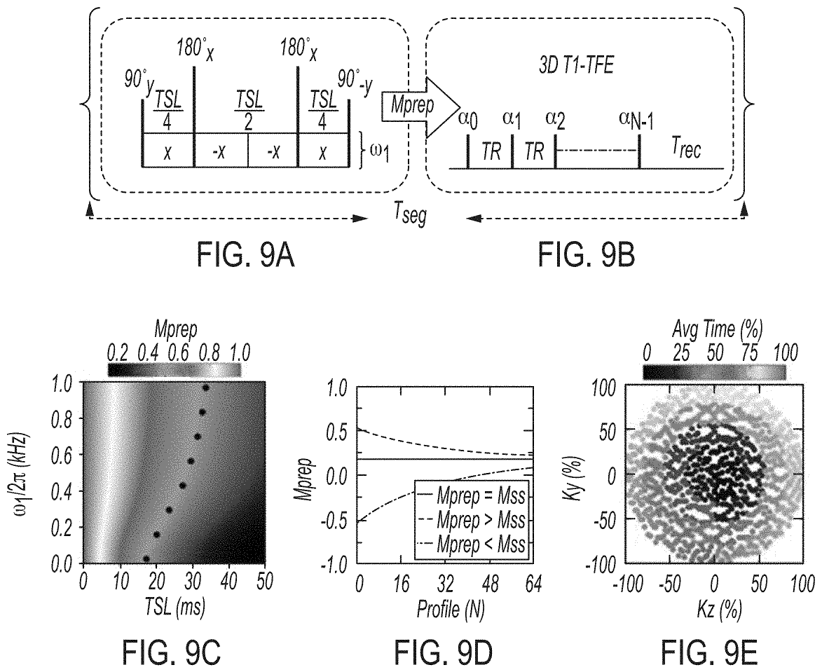

That is, a new spin-lock (“SL”) method has been proposed for quantitative R_{1ρ} dispersion of human knee articular cartilage (FIG. 9A), which is less prone to B₀ and B₁ field artifacts for a broad range of ω₁/2π settings as demonstrated by Bloch simulations, phantom imaging, and in vivo experiments. The enhanced robustness of this new SL method is derived from the fully refocused prepared R_{1ρ} magnetization (Mₚᵣₑₚ) by two self-compensated refocusing pulses even when they are not exactly equal to 180°. Therefore, Mₚᵣₑₚ from this new SL approach should become larger than those with previous methods when B₁ field is not uniform.

The differently prepared Mₚᵣₑₚ evolution towards steady-state during turbo-FLASH imaging readout can be translated into a varying k-space filtering effect, resulting in a biased R_{1ρ}. An image will be completely free of such systematic errors only if the k-space filter remains constant for all k-space lines. One approach to achieving this goal is to tailor Mₚᵣₑₚ into a narrow range; however, this reduced dynamic range in Mₚᵣₑₚ could inevitably introduce additional uncertainty in determining R_{1ρ} when fitting the near constant R_{1ρ}-weighting to an exponential relaxation decay model.

In particular, the present disclosure provides an efficient and robust R_{1ρ} dispersion imaging protocol for human knee cartilage clinical studies. Specifically, the present disclosure provides a novel method to prepare a near constant Mₚᵣₑₚ by tailoring both SL RF duration (TSL) and ω₁/2π, and the limited dynamic range in Mₚᵣₑₚ will be expanded by exploiting extra information derived from the magic angle (MA) location or when ω₁/2π=∞. Hence, the present disclosure provides an efficient and robust method for quantitative R_{1ρ} dispersion imaging of human knee articular cartilage. Advantageously, this method allows comparable image quality to be obtained with about a 30% reduction in scan time compared to standard R_{1ρ} mapping.

Systems and Methods for Analyzing Ordered Tissue to Calculate an Orientation-Independent Order Parameter S that is Sensitive to the Collagen Microstructural Integrity in Cartilage

### Theory

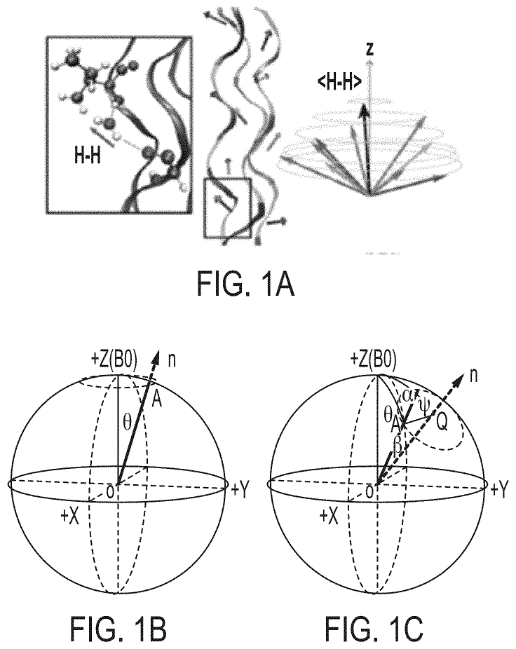

The transverse relaxation R₂ of water proton in cartilage is largely induced by a dominant intramolecular dipolar interaction (R₂^{dd}) and an increasing chemical exchange effect (R₂^{ex}) as the static magnetic field B₀ increases. Specifically, R₂^{dd} stems from preferentially orientated water in collagen, where the bound water is fixed by two hydrogen bonds connecting with neighboring chains in triple-helix interstices. As a result, an effective <H—H> dipolar interaction vector tends to align along the principal axis of collagen fibers as shown in FIG. 1A, which was revealed by a molecular dynamics simulation study on a hydrated collagen model peptide. On the other hand, the secondary R₂^{ex} is typically attributed to a fast chemical exchange between hydroxyl (—OH) protons in bulk water and in PG (mostly glycosaminoglycan, GAG) with different chemical shifts (Δω≈1 ppm). Taking together, R₂ can be quantified by three characteristic contributions as expressed in EQUATION 1, where R₂^{dd} has been divided into an isotropic R₂ⁱ and an anisotropic R₂^{a}(θ).

These three contributions to R₂ can be categorized into different two groups, depending on their orientation dependences or the time scales of water-protein interactions. For instance, R₂^{a}(θ) is orientation-dependent in contrast to R₂ⁱ and R₂^{ex}. In the meantime, R₂^{ex} and R₂^{a}(θ) are only sensitive to slow time scale interactions and thus can be suppressed in R_{1ρ} measurements depending on the spin-lock RF strength (ω₁) and the relevant correlation time (τ_{b}) and chemical exchange time (τₑₓ) for CA− and GAG− water interactions as given in EQUATION 2.

$$\begin{matrix}{R}_{2} = {R}_{2}^{i} + {R}_{2}^{a} ( θ ) + {R}_{2}^{e x} & ( 1 ) \\ {R}_{1 ρ} = {R}_{2}^{i} + \frac{{R}_{2}^{a} ( θ )}{1 + 4 {ω}_{1}^{2} {τ}_{b}^{2}} + \frac{{R}_{2}^{e x}}{1 + 4 {ω}_{1}^{2} {τ}_{e x}^{2}} & ( 2 )\end{matrix}$$

Note, τₑₓ⁻¹ is redefined here as the average, instead of the sum, of the rate constants of the forward (k_{AB}) and reverse (k_{BA}) reactions. Apparently, R_{1ρ} will turn respectively into R₂ or R₂ⁱ when ω₁ is absent or sufficiently strong (i.e. ω₁>>τ_{b}⁻¹ and τₑₓ⁻).

When it becomes significant, R₂^{ex} can be further separated from R₂^{dd} based on either the former's B₀² dependence or the latter's orientation dependence. R₂^{ex} is normally quantified with p_{A}p_{B}Δω²(2πₑₓ), with p_{A/B} and Δω representing molecular fractions and an angular chemical shift difference in and between A (—OH in water) and B (—OH in GAG) states. On the other hand, R₂^{a}(θ) can be written as R₂^{a} [symbol] 3 cos² θ−1 [symbol] ²/4, with an angle θ formed between B₀ (+Z) and an effective residual dipolar interaction vector (\vec{OA}) along a principal axis (\vec{n}) in collagen fibers as depicted in FIG. 1B. By comparing R₂ or R_{1ρ} measured at two different B₀ (e.g. 3T vs. 7T), R₂^{ex} can be readily separated because R₂^{dd} is basically independent of B₀. Alternatively, if multiple orientation-dependent R₂ measurements are available at one B₀, R₂^{a}(θ) can be removed first from R₂ using EQUATION 1 and R₂^{ex} can then be detached further from R₂ⁱ by a specific R_{1ρ} dispersion (EQUATION 2) at the magic angle orientations where R₂^{a}(θ) becomes zero. It is worth noting that R₂^{ex} could only become relevant at higher magnetic fields (B₀>3T) or around the locations with R₂^{a}(θ) approaching zero such as in the cartilage transitional zone or close to the magic angle orientations for collagen fibers.

Regarding the water-CA interactions responsible for R₂^{a}(θ), it seems more realistic and revealing to characterize \vec{OA} in a dynamic picture using an axially symmetric molecular reorientation model as shown in FIG. 1C, where \vec{OA} rapidly rotates about a symmetric axis \vec{n} at an angle of β, and \vec{n} makes an angle of α with B₀. Because of this rapid molecular reorientation with a characteristic small correlation time τ_{∥}, the orientation-dependent term [symbol] 3 cos² θ−1 [symbol] in R₂^{a}(θ) will be mathematically transformed into [symbol] 3 cos² β−1 [symbol] (3 cos² α−1)/2, where angle brackets [symbol] . . . [symbol] indicate a time or an ensemble average. As a result, R₂^{a}(θ) can be quantified by two different terms that are grouped in two pairs of curly brackets in EQUATION 3.

$${R}_{2}^{a} ( θ ) = \{ \frac{3}{2} {( d \frac{〈 3 {cos}^{2} β - 1 〉}{2} )}^{2} \} \{ {( \frac{1 - 3 {cos}^{2} α}{2} )}^{2} {τ}_{⊥} \} \qquad ( 3 )$$

The first term contains a scaled dipolar interaction constant Sd, with a scaling factor S defined as [symbol] 3 cos² β−1 [symbol] /2 and d a constant of \sqrt{3/10}(μ₀/4π) (γ²hr⁻³), e.g. d=1.028*10⁵ (s⁻¹) with a distance r of 1.59 (Å) between two proton nuclei in water. In literature, S was referred to as an order parameter—a measure of water molecular reorientation restrictions. For instance, S could have become zero had the bound water been orientated randomly in collagen. The second term is directly related to the well-known magic angle effect, where the correlation time τ_{⊥} characterizes a much slower molecular reorientation (i.e. τ_{⊥}>>τ_{∥}) about an axis perpendicular to \vec{n}, and is considered to be associated with different processes of breaking and reforming the hydrogen bonds mediated by the bound water in collagen triple-helix interstices. For this oversimplified model, only one correlation time τ_{⊥} is adequate to characterize the bound water anisotropic molecular motion.

It is noteworthy that EQUATION 3 can be derived by simplifying a general form of anisotropic R₂ equation by assuming an axially symmetric model for a preferential water orientation in collagen. It is also worth pointing out that the rotational axis (\vec{n}) relative to B₀ (i.e. α) could be arbitrarily manipulated; however, the intrinsic bound water's bonding property β or S should not be altered in the orientation-dependent MR relaxation studies on cartilage. This observation basically suggests that R₂^{a}(α) should be proportional to τ_{b}(α) regardless of collagen orientations, with τ_{b}(α) representing τ_{⊥}(1−3 cos² α)²/4. As a result, an orientation-independent order parameter S can be calculated using EQUATION 4 if R₂^{a}(α) and τ_{b}(α) could be derived from R_{1ρ} relaxation dispersion.

$$S = \sqrt{\frac{2}{3 {d}^{2}} \frac{{R}_{2}^{a} ( α )}{{τ}_{b} ( α )}} \qquad ( 4 )$$

The uncertainty in S can also be determined if the measurement errors in R₂^{a}(α) and τ_{b}(α) are available using the standard error propagation formulas. Note, the different orientation symbol (α vs. θ) is irrelevant in EQUATION 4.

### Methods

### MRI Acquisition

Seven orientation-dependent R₂(θ) and standard R_{1ρ} (θ, ω₁) dispersion (θ≈n*15°, n=0-6; ω₁/2π=0.25. 0.5. 1.0. 2.0 kHz) measurements on bovine patellar cartilage-bone samples (n=4) were performed at 9.4T by others, and the corresponding relaxation depth-profiles were publicly available and used in this study. More details can be found in the original publication.

One human volunteer's right knee was studied with R_{1ρ} (1/T1ρ) dispersion in the sagittal plane using a 16-ch T/R knee coil on a research-dedicated Philips 3T MR scanner. 3D T1ρ-weighed images with varying spin-lock (a) times (TSL=1, 10, 20, 30 and 40 ms) were acquired with a SL-prepared T1-enhanced 3D TFE pulse sequence, where five SL RF pulse strengths (ω₁/2π=0.125, 0.25, 0.5, 0.75, 1.0 kHz) were used for different R_{1ρ} mappings. The acquired voxel size was 0.40*0.40*3.00 mm³ and interpolated to 0.24*0.24*3.00 mm³ in the final reconstructed images. Total scan duration was about 45 minutes.

### Rip Dispersion Modeling

### Bovine Patellar Cartilage

The orientation-depth maps of R₂(θ) and R_{1ρ}(θ, ω₁) were reproduced using a slightly modified matlab script provided in the original study, with a linear interpolation replaced by a spline version to avoid undefined profiles on the map edges. This study focused only on the deep cartilage where average relaxation rates were calculated for further analysis. The deep zone was defined within a normalized depth range between 40% and 80% from the articular surface.

The chemical exchange contribution (R₂^{ex}) was first separated based on the orientation-dependence of R₂(θ) and the specific dispersion of R_{1ρ}(θ_{MA}, ω₁). In modeling R₂(θ), the sample orientation θ was allowed to float within a limited range of [−30°, 30°] to account for the potential errors in positioning samples and the actual orientation deviations of collagen fibers, Then, R_{1ρ} (θ, ω₁), excluding R₂^{ex}, was fitted to a function of A+R₂^{a}(θ)/(1+4ω₁²τ_{b}²(θ)) for different θ, where A, R₂^{a}(θ) and τ_{b}(θ) were model parameters. Subsequently, S was derived from each pair of the fitted R₂^{a}(θ) and τ_{b}(θ) at different orientation B not close to B_{MA} (i.e. <50° or 35°). Finally, average S and its standard deviation for each bovine patellar sample were calculated.

TABLE 1 tabulates the categorized R₂ absolute (1/s) and relative (%) relaxation rates, the fitted magic angles θ_{MA}, τₑₓ (μs), the average [symbol] R₂^{a}(θ) [symbol] and the average [symbol] τ_{b} [symbol] in terms of the data ellipse centroids and S for each sample. The model parameter ranges were constrained in in nonlinear χ²-based curve-fittings: R₂^{a}(θ)=[0, 300] (1/s); R₂ⁱ and R₂^{ex}=[0, 30] (1/s); τₑₓ and τ_{b}=[10¹, 10³] (μs). If the determined model parameters were equal to the predefined limits or their relative errors were large than 100%, they had been excluded for further analysis.

### Human Knee Cartilage

3D R_{1ρ}-weighted images were first co-registered following an established protocol, and R_{1ρ} pixel maps with different ω₁/2π were produced by curve-fittings to a simple exponential decay model (two parameters). Next, the angular and radial segmentations were performed on the femoral, tibial and patellar cartilage and ROI-based three parameters (R₂ⁱ, R₂^{a}(θ) and τ_{b}(θ)) were fitted using EQUATION 2 with R₂^{ex} set to zero, and average order parameter S was reported for all three cartilages in TABLE 2 including the descriptive statistics for varying R_{1ρ} dispersion and modeling parameters as well. As described above, the ranges of the model parameters for R_{1ρ} dispersion and the criteria in selecting the accepted fitted parameters were the same as those used in bovine cartilage samples.

### Statistical Analysis

The differences and correlations between any two relaxation metrics were quantified using the Student's paired t-test (a two-tail distribution) and the Pearson correlation coefficient (r), with the statistical significance considered at P<0.05. Inter-group comparisons were evaluated using box-and-whisker plots and histograms, and the potential correlations between any two parameters were visualized in scatterplots with 95% confidence level data ellipses overlaid. All measurements are shown as mean±SD unless stated otherwise. All image and data analysis were performed using in-house software developed in IDL 8.5 (Exelis Visual Information Solutions, Boulder, Colo.).

### Results

### Bovine Patellar Cartilage

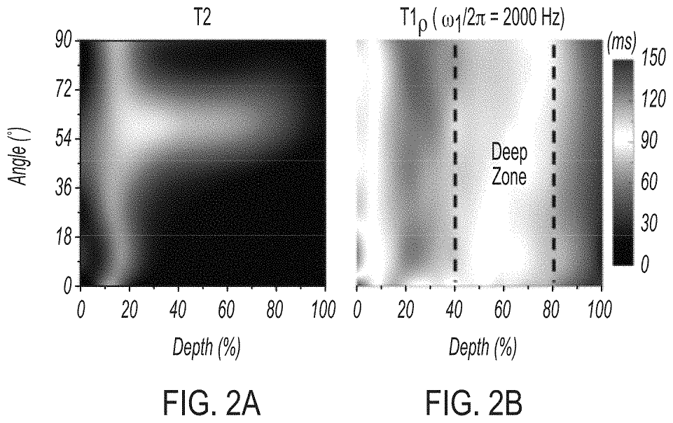

FIG. 2 reproduces the original orientation-dependent relaxations T₂ (1/R₂) and T_{1ρ} (1/R_{1ρ}) depth-profiles without (A) and with a spin-lock (SL) RF (ω₁/2π=2000 Hz) (B) from one bovine cartilage sample (B1S2). The magic angle effect can be easily appreciated in the deep zone when the sample orientated near 60° relative to B₀ (A); however, this intrinsic R₂ anisotropy was mostly suppressed when using a stronger SL RF in R_{1ρ} measurements (B).

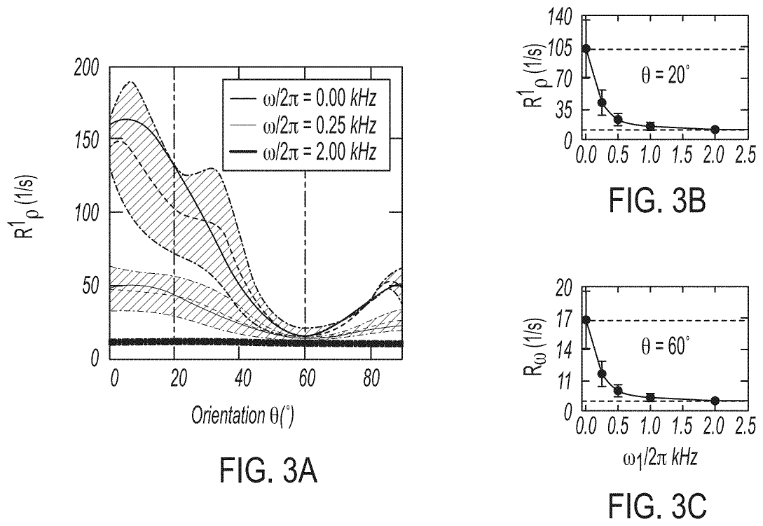

FIG. 3 provides an example of R₂ (1/s) partition and R_{1ρ} (1/s) dispersion for the same sample B1S2. Specifically, R₂ was separated into an anisotropic R₂^{a} (147.5±2.4) and an isotropic part composed of R₂ⁱ and R₂^{ex} (i.e. R₂ⁱ+R₂^{ex}=16.0±0.4) by an orientation dependence fitting (red solid line) (A), with the fitted magic angle θ_{MA} equal to 59.6±0.3° . In this subplot, two different orientation-dependent R_{1ρ} relaxation profiles with ω₁/2π of 0.25 (green solid line) and 2.0 kHz (blue solid line) were also included.

To further separate R₂^{ex} from R₂ⁱ, a particular R_{1ρ} dispersion fitting was carried out at θ_{MA} (C), resulting in the fitted R₂ⁱ of 10.4±0.2 (1/s), R₂^{ex} of 5.6±0.2 (1/s) and τₑₓ of 161.7±12.9 (μs), respectively. A typical modeling of R_{1ρ} dispersion (θ=20°), excluding R₂^{ex}, is also presented (B) with the fitted R₂ⁱ of 11.3±3.3 (1/s), R₂^{a}(θ) of 86.3±5.3 (1/s) and τ_{b}(θ) of 459.0±28.7 (μs), respectively. These exemplary analyses indicate that an anisotropic R₂^{a} was the dominant (90%) contribution to R₂, and R_{1ρ} dispersion was orientation-dependent.

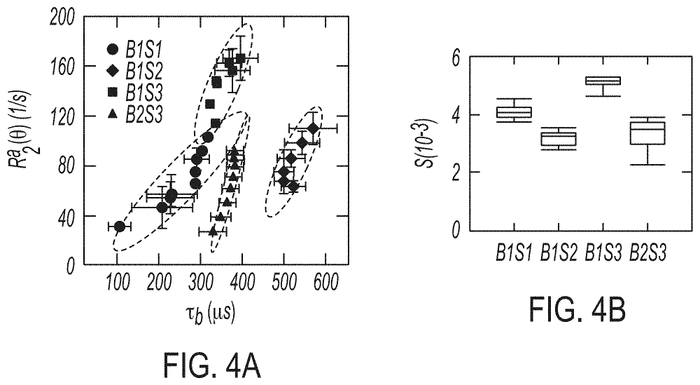

TABLE 1 summarizes the average R₂ partitions, R_{1ρ} dispersion modeling parameters, average [symbol] τ_{b}(θ) [symbol] and average [symbol] R₂^{a}(θ) [symbol] and the derived order parameter S for each of four samples, showing that the chemical exchange effect (R₂^{ex}) contributed about 3% to R₂ and the determined magic angle θ_{MA} (64.4±8.9°) deviated from an assumed 54.7°. More importantly, the derived τ_{b}(θ) demonstrated a significantly high correlation (r=0.89+0.05, P<0.05) with the corresponding R₂^{a}(θ) as predicated despite varying linear relationships for different samples as shown in FIG. 4A. As a result, the derived average order parameters S (10⁻³) varied from 3.15±0.28 (B1S2) to 5.08±0.24 (B1S3) as shown in the box-and-whisker diagrams in FIG. 4B and listed in TABLE 1.

### Human Knee Cartilage

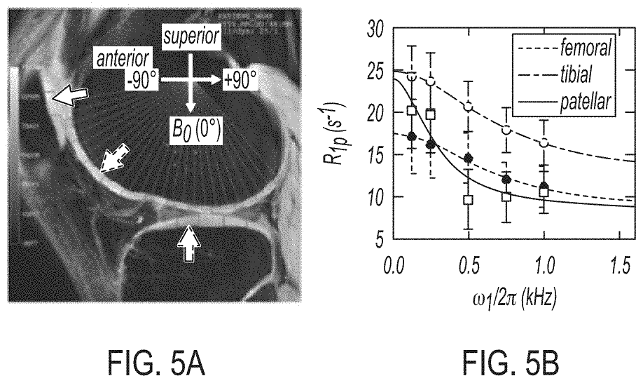

FIG. 5B presents three ROI-based (indicated by colored arrows) R_{1ρ} dispersions (indicated by colored solid lines) in the femoral (red), tibial (green) and patellar (blue) cartilage on one R_{1ρ}-weighted image as shown in FIG. 5A. The observed largest and the smallest R₂ (ω₁/2π=0) from the tibial and the femoral cartilage were in good agreement with the theoretical predication, as the deep collagen fibers within these two ROIs were nearly parallel and at the magic angle to B₀.

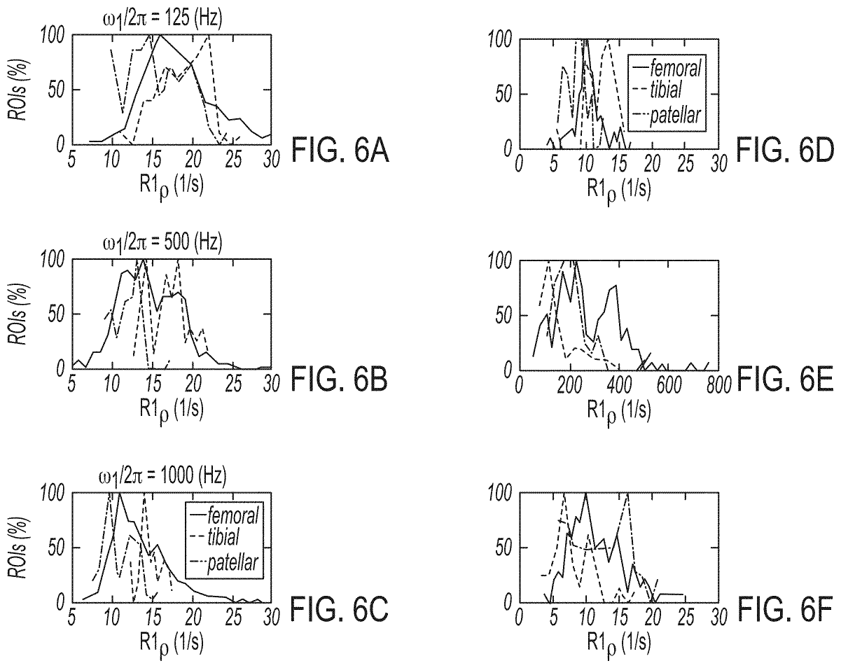

FIG. 6 shows histogram comparisons of the measured and the modeled R_{1ρ} dispersions, with ω₁/2π=125 Hz (A), 500 Hz (B) and 1000 Hz (C) and fitted R₂ⁱ (D), τ_{b} (E) and R₂^{a}(θ) (F) in the femoral (red), tibial (green) and patellar (blue) cartilage of whole human knee, and the corresponding descriptive statistics of these presented data are tabulated in TABLE 2. As the spin-lock RF strength increased from 125 to 1000 Hz, the absolute values (1/s) and anisotropies of R_{1ρ} decreased from 19.4±5.7 to 13.5±3.4 in the femoral (red solid lines), from 19.0±3.3 to 14.1±1.7 (green dash-dot lines) in the tibial and from 16.9±3.4 to 11.4±1.6 (blue dashed lines) in the patellar cartilage, as indicated by the left shifted and narrowed histograms (A-C). On the other hand, the fitted R₂ⁱ became clustered within limited ranges (D) and the derived τ_{b} were positively correlated (r=0.51, P<0.01) with R₂^{a}(θ) in all three cartilages (E-F).

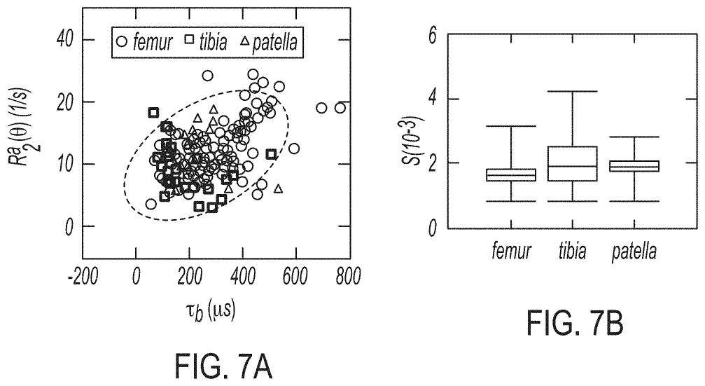

FIG. 7 presents a scatterplot (A) between the fitted orientation-dependent τ_{b} and R₂^{a}(θ) for the femoral (red circles), tibial (green squares) and patellar (blue triangles) cartilage, and a box-and-whisker diagram for the derived order parameters S (B) for each cartilage in human knee. A summary of the descriptive statistics of the measured and the modeled R_{1ρ} dispersions is listed in TABLE 2. In general, the estimated R₂ⁱ (1/s) was comparable (i.e. ˜10.8) in all cartilages; more importantly, the derived average order parameters S (10⁻³) for three different cartilage was similar (i.e. ˜1.84) in spite of varied R₂ relaxation anisotropies.

### Discussion

### General Comment

In the present disclosure, a theoretical framework to derive an orientation-independent order parameter S for the bound water in collagen through R_{1ρ} dispersion is provided and corroborated on bovine patellar cartilage samples at 9.4T and one live human knee at 3T. The proposed order parameter S can be considered as an intrinsic MR probe reflecting the microstructural integrity of highly organized tissues. Since the developed method is not only limited to cartilage, it could be extended to other structured tissues in clinical studies. For example, R_{1ρ} dispersion has been used for characterizing myocardial fibrosis and the relaxation mechanisms underlying the proposed novel non-contrast cardiac magnetic resonance (CMR) index could be elucidated if using the similar approaches as discussed in the present disclosure.

The present disclosure describes the first attempt to separate the magic angle effect from MR relaxation measurements and yet to retain the most relevant water bonding information in highly organized tissue. To date, the compositional MR relaxation study on ordered tissue was only useful for longitudinal investigations in which the magic angle effect would be automatically decoupled if the tissue at the same location is considered. With the proposed method, however, it is possible to make the reliable diagnosis on the focal degenerative changes relative to other intact cartilage on the same knee, which could have a great impact on the diagnosis of early cartilage degeneration in clinical practice.

### Anisotropic Molecular Reorientation

Five different correlation times are generally required to adequately characterize an anisotropic molecular motion according to the classical NMR relaxation theory; however, the number of these correlation times can be reduced to three if an axially symmetric model is assumed. In this scenario, the three pertinent correlation times will be constructed from two independent ones (e.g. τ_{∥} and τ_{⊥}) that characterize the molecular reorientations about and perpendicular to the axially symmetric rotational axis. If an additional assumption is made such that τ_{⊥}>>τ_{∥}, as discussed in the present disclosure, the only relevant correlation time will be the much slower one (τ_{⊥}); in other words, an anisotropic molecular reorientation with an oversimplified axially symmetric model can be treated as a conventional isotropic molecular rotation characterized with a large effective correlation time.

Accordingly, R₂ and R_{1ρ} will become sensitive to these slow time scale molecular interactions between water and collagen but not for R₁, which depends only on fast time scale molecular motions. It cannot be stressed enough that R₂ (ω₁/2π=0) is the most sensitive metric for the slow time scale interactions given various R_{1ρ} relaxation dispersions. Recently, a composite relaxation R₂-R_{1ρ} was proposed as an early predictor of cartilage lesion progression, which simply states that R₂ is more sensitive than R_{1ρ} regardless of the exact relaxation mechanism for the slow time scale molecular interactions. It is also worth mentioning that the relative change rather than the absolute value of R_{1ρ} should be used to characterize cartilage degeneration. This interpretation differs from some previous reports that R_{1ρ} itself was considered as an important MR biomarker for early cartilage degeneration.

### An ARCADE Model for Collagen Fibers

The collagen fibers in articular cartilage are commonly categorized into a superficial (parallel), a transitional (arcading) and a deep (perpendicular) zone based on the preferential direction of the fibers relative to cartilage surface. Had the cartilage surface been perpendicular to B₀ and the collagen fibers in the deep zone been perpendicular to the cartilage surface, the minimum R₂ should have been detected at the magic angle θ_{MA} of 54.7°. However, an average θ_{MA} estimated in this study was offset by about 10° from the expected value. These unexpected observations could be partially explained by either that the cartilage surface was not exactly perpendicular to B₀ or that the collagen fibers were not exactly perpendicular to the cartilage surface. In either case, the routine experimental setup for relaxation measurements would become tedious if consistent results are expected from repeated scans. Nevertheless, the developed method provided in the present disclosure could make such relaxation studies less demanding as the orientation-dependent factor has been taken out of the equation in the proposed order parameter S.

Order Parameters from Normal Cartilage

In this study, the derived S from bovine patellar cartilage samples had demonstrated both intra- and inter-sample variabilities (FIG. 4B). For a particular sample, S could be subjected to a limited number of orientation-dependent measurements or an insufficient signal-to-noise ratio (SNR) of R₂^{a}(θ) when close to θ_{MA}. As stated in the original paper, the exact age of the animals was not known and thus the age-dependent factor might have contributed to the altered collagen structures revealed by various S among samples. On the other hand, the hydration differences in prepared samples could lead to various water bonding properties and thus altered order parameters S. In his pioneering paper, Berendsen clearly demonstrated that water bonding to collagen (from bovine Achilles tendon), in terms of water proton resonance doublet splitting, depended heavily on the hydration level, with S (10⁻³) decreased approximately from 45 to 26 when the relative humility (hydration) increased from 32% to 90%.

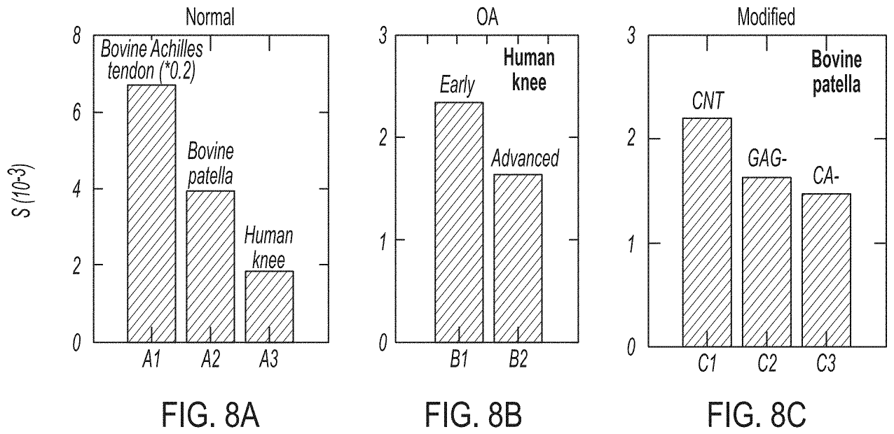

It is not surprising that S could be indicative of varying biomechanical properties for different cartilage, given the molecular basis of the bound water in collagen. For instance, S from an asymptomatic human knee cartilage was estimated to about 2.0*10⁻³ (FIG. 7B), compared to about 4.0*10⁻³(FIG. 4B) in bovine knee patellar cartilage samples in this study. However, these two order parameters S were much smaller than that obtained from the hydrated bovine Achilles tendon (˜35.0*10⁻³ at ˜25% hydration) as compared in FIG. 8A.

Order Parameters from Modified and OA Cartilage

For the very reason underlying the water bonding, the proposed order parameters could be an essential MR biomarker for early cartilage degeneration. This potential utility was documented with one R_{1ρ} dispersion study at 9.4T on both enzymatically modified bovine patellar cartilage samples and human tibial cartilages with early and advanced OA. In that work, the derived correlation times τ_{b} was investigated and suggested as a fundamental biophysical MRI contrast. As explained in the present disclosure, τ_{b} and anisotropic R₂ are not only correlated with each other but also dependent on the same geometric factor.

As a result, the corresponding order parameters S could be estimated for human OA cartilage and biochemically degraded bovine cartilage samples as shown in FIG. 8, where S (10⁻³) from early OA was larger (i.e. 2.36>1.64) than that from advanced OA samples (B), and decreased sequentially from the control (CNT=2.02) to the GAG− depleted (GAG−=1.64) to the collagen-depleted (CA−=1.47) samples (C). These order parameters S were derived according to the reported τ_{b} (μs) and an estimated R₂^{a} (1/s) from the graphs for the whole (100%) depth cartilage, e.g. [R₂^{a}, τ_{b}]=[38, 432] and [27, 634] for early and advanced OA samples; [20, 310], [16, 374] and [12, 350] for CNT, GAG− and CA− samples. If the superficial zone (5% depth) was considered, the observed S trend would be even more clear; specifically, S (10⁻³) would become 1.82 vs.1.16 for early and advanced OA, and 1.16 vs. 1.10 vs. 0.72 for CNT, GAG− and CA− samples. These ex vivo results strongly support the argument that the proposed order parameters S could be a promising MR biomarker for the integrity of the collagen microstructure in cartilage.

### Future Work

A judicious design for an efficient R_{1ρ} dispersion imaging is conceivable in future research, which can not only reduce potential involuntary motion artifacts but also facilitate the implementation of the proposed method into a routine clinical imaging protocol. One possible approach could be a constant time R_{1ρ} dispersion in which the varied parameter would be a spin-lock RF amplitude instead of its duration. Once an efficient R_{1ρ} dispersion protocol becomes available, other highly organized tissues (e.g. myocardium) could be explored to elucidate the relevant relaxation mechanism in the diseased state (e.g. fibrosis) and thus the specific structural protein could be clinically investigated.

### Conclusion

The results from applying this new concept to both ex vivo and in vivo articular cartilage studies demonstrate that an orientation-independent order parameter S that is sensitive to the microstructural integrity of highly ordered tissues can be established from R_{1ρ} dispersion. It is foreseen that the developed unique approach will broaden the current spectrum of the compositional MR imaging applications in clinical practice.

### Efficient and Robust R_{1ρ} Dispersion Mapping of Human Knee Cartilage Using Tailored Spin-Locking in an Optimized 3D Turbo-FLASH Sequence

### Methods

### Spin-Lock and Turbo-FLASH Sequence

### A Fully-Refocused Spin-Lock Preparation

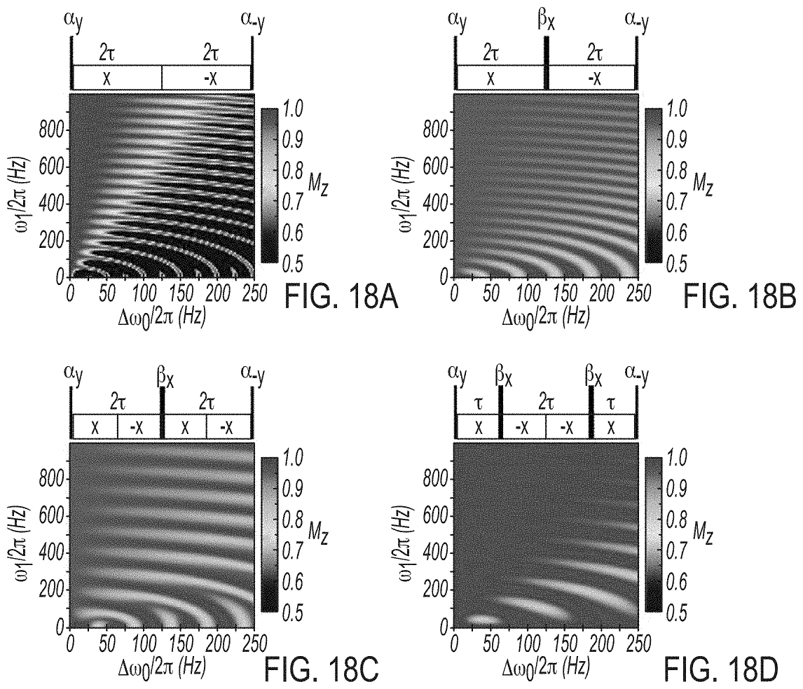

As shown in FIGS. 9A and 18D, the double refocusing RF pulses (β) in the proposed SL scheme, unlike none or only one in previous methods (e.g., as shown in FIGS. 18A-C), can fully refocus the chemical shift (Δω₀) artifacts originated from non-uniform B₀ even when β is not exactly equal to 180° due to B₁ inhomogeneity. Essentially, the proposed scheme was a fully-refocused hybrid-echo approach, comprising two pairs of antiphase rotary-echo pulses with each flanking one refocusing pulse. The previous methods discussed here included the rotary-echo approach to mitigating B₁ artifacts (e.g., as shown in FIG. 18A), a combined rotary-and spin-echo (i.e. hybrid-echo) scheme (e.g., as shown in FIG. 18B) for removing both B₀ and B₁ artifacts when using a lower ω₁/2π (e.g. 27 Hz) at 3T, and a modified hybrid-echo method (see FIG. 18C) for a higher ω₁/2π (e.g. 1 kHz) at 7T.

Bloch simulations using various rotation matrices were carried out to evaluate the improved SL performance using a relatively broad range of ω₁ and Δω₀ suitable for human knee cartilage imaging at 3T. Specifically, ω₁/2π increased evenly from 0 to 1000 Hz and Δω₀/2π from 0 to 250 Hz in 101 steps to simulate spin dynamics starting from an equilibrium state. Since only the longitudinal component of the prepared magnetizations will be mapped out by the FLASH imaging sequence, the transverse components were thus excluded for further considerations. In these simulations, the nominal flip angle (FA) α and β were scaled down 90% to mimic inhomogeneity reported for human knee cartilage imaging at 3T. Also, any relaxation effects during RF flipping, refocusing and SL were not considered, i.e. α and β were treated as hard pulses.

### An Optimal FA for Turbo-FLASH Sequence

The steady-state longitudinal magnetization (Mₛₛ) from magnetization-prepared spoiled FLASH sequence does not depend on an initial condition (Mₚᵣₑₚ), but rather is a function of the constant excitation FA of α₀, repetition time TR, and longitudinal relaxation time constant, T₁, of the tissue, as shown by EQUATION 5,

$${M}_{SS} = {M}_{0} \frac{( 1 - {E}_{1} )}{( 1 - {E}_{1} cos {α}_{0} )} \qquad ( 5 )$$

where M₀ is the magnetization in an equilibrium state, and E₁=exp (−TR/T1). The transient magnetization (M_{N}) immediately before an excitation RF pulse, α_{N}, could be written as EQUATION 6,

M_{N}=M_{SS}+(Mₚᵣₑₚ−M_{SS})(E₁ cos α₀)^{N} (6)

where Mₚᵣₑₚ is the prepared R_{1ρ}-weighted magnetization (normalized), ranging potentially from −1 to 1 depending on the phase of the flip-back RF pulse as well as TSL and ω₁/2π. Hence, an average of the measurable magnetization (M̅) could be calculated as the sum per shot (or segmentation), i.e. as a function of α₀,

M̅={sin α₀/(N−1)}Σ₀^{N-1}M_{N} (7)

Consequently, an optimal α₀ for each Mₚᵣₑₚ could be identified given the knowledge of N, TR and T₁. In this work, simulations were performed with the following parameters: TR=6.8 ms and T₁=1240 ms, α₀ ranging from 0° to 24° and Mₚᵣₑₚ from 0 to 100% for each N (i.e. 32, 64, 96, 128). In vivo experiments were conducted on the first subject's left knee to validate the predicted optimal FA (see below).

### Quantitative R_{1ρ} Dispersion Imaging

### Tailored Constant R_{1ρ} Weighting

The signal strength in R_{1ρ}-weighted cartilage image could be expressed by EQUATIONS 8-9, assuming a negligible chemical exchange contribution to R_{1ρ} at 3T.

$$\begin{matrix}S ( T SL , {ω}_{1} ) = {S}_{0} exp ( - {R}_{1 ρ} * TSL ) & ( 8 ) \\ {R}_{1 ρ} = {R}_{2}^{i} + \frac{{R}_{2}^{a} ( θ )}{1 + 4 {ω}_{1}^{2} {τ}_{b}^{2}} & ( 9 )\end{matrix}$$

Here, R₂ⁱ stands for a non-specific isotropic relaxation component, R₂^{a}(θ) for a specific anisotropic contribution and τ_{b} for the corresponding slow (˜μs-ms) reorientation correlation time for those motion-restricted water molecules in collagen. Generally, R₂^{a}(θ) is written as R₂^{a} [symbol] 3 cos² θ−1 [symbol] ²/4, with θ an angle between the collagen fiber direction and B₀; thus, R₂^{a}(θ) will become zero when θ is at the MA of 55°.

The prepared SL magnetization, Mₚᵣₑₚ=S(TSL, ω₁)/S₀, is determined by the user-defined parameters TSL and ω₁; thus, a near constant Mₚᵣₑₚ could be generated by imultaneously increasing or decreasing both parameters, given that other related parameters (R₂ⁱ, R₂^{a} and τ_{b}) are constant. Eight different combinations of TSL and ω₁ values for three Mₚᵣₑₚ preparations (i.e. 50%, 60% and 70%) were listed in TABLE 3, with an assumption of R₂ⁱ=R₂^{a}=20 (1/s) and τ_{b}=300 (μs).

According to EQUATION 9, R_{1ρ} will become R₂ⁱ when θ=55° or ω₁=∞. This fact was exploited to increase the dynamic range for the constant Mₚᵣₑₚ preparation, where the signal derived with θ=55 could be considered as that with ω₁=∞. This extra information is referred to as an internal reference (REF), i.e. REF1 for θ=55 and REF2 for ω₁=∞.

Simulated Quantitative R_{1ρ} Dispersion with Noise

Monte Carlo simulations were performed to evaluate the accuracy and precision of R_{1ρ} dispersion quantification with and without an REF. An R_{1ρ} dispersion profile was generated based on EQUATIONS 8-9 following the protocols listed in TABLE 3, with S₀=100, R₂ⁱ=R₂^{a}=20 (1/s), TSL ranging from 9 to 32 ms, ω₁/2π from 0 to 1000 Hz and τ_{b}=300 (μs). As shown before (5), an orientation-independent order parameter S (10⁻³) can be determined given the values of R₂^{a} and τ_{b}, and it was 2.052 herein when using a constant K of 1.05610¹⁰ (s⁻²) in S=\sqrt{(R₂^{a}/τ_{b})(1/1.5K)}.

Next, the simulated data were contaminated with Gaussian noise leading to 9 signal-to-noise ratios (SNRs) from 20 to 100. Here, the SNR was defined as S₀/σ, with σ standing for the standard deviation (SD) of the Gaussian noise. These defined noises were generated from normally distributed random numbers with zero mean and different variance depending on SNR. The noisy R_{1ρ} dispersion profile was generated 1000 times for each SNR with Mₚᵣₑₚ=50%, 60%, 70%, respectively. An REF data were calculated for each of eight TSL values with ω₁=∞. Thus, each Mₚᵣₑₚ group would have had 16 different R_{1ρ}-weighted datasets had the REF data been used. In order to assess to what extent a biased REF could have compromised R_{1ρ} dispersion quantification in a realistic scenario, a noiseless dataset was prepared with S₀=100, R₂ⁱ=15 (1/s), R₂^{a}=20 (1/s) and τ_{b}=200, and then an erroneous REF was created using a biased R₂ⁱ with a relative uncertainty (ΔR₂ⁱ) ranging from −100% to +100%.

From these 1000 simulations, the mean and SD of each of the fitted R_{1ρ} dispersion parameters were calculated. The accuracies of these estimated parameters were evaluated in terms of the root mean square error (RMSE) defined by

{square root over (Σᵢ₌₀^{j}{(P_{fit}ⁱ−Pₜᵣᵤₑ)/Pₜᵣᵤₑ}²/(j−1))}*100%

where P_{fit}ⁱ and Pₜᵣᵤₑ were the fitted and the true (input) values, and j was 1000 in this study. Here, the SD of the fit was considered as the fitting precision.

### In Vivo MR Imaging

Three consented volunteers part of an IRB-approved clinical study evaluating post-traumatic OA after anterior cruciate ligament (ACL) surgical reconstruction were recruited and their asymptomatic knees were investigated using the developed R_{1ρ} dispersion imaging protocol (see below). The first subject had a bilateral knee scanned using Mₚᵣₑₚ of 50%, 60% and 70%, while the second and the third subjects only had a single knee imaged using Mₚᵣₑₚ of 60%. In addition, several extra R_{1ρ} imaging scans (see below) were collected to confirm the predicted optimal FA, and to compare the derived R_{1ρ} values with those reported in the literature. Particularly, the second and the third subjects had their knees re-imaged 3 months later using both the developed (i.e. improved) R_{1ρ} dispersion and standard (i.e. original) R_{1ρ} mapping protocols.

### Quantitative R_{1ρ} Dispersion Imaging Protocol

Eight constant R_{1ρ}-weighted images for each of three Mₚᵣₑₚ preparations were acquired with an optimized 3D turbo-FLASH sequence (see FIGS. 9A-9B) in which the tailored TSL and ω₁ values can be found in TABLE 3. The other relevant acquisition parameters were as follows: SL 90°/180° RF durations=0.25/0.5 (ms); FOV=130*130*96 (mm³); acquired voxel size=0.6*0.6*3.0 (mm³); number of slices=32; Compressed SENSE factor=2.5; fat suppression=“binomial (1-2-1) pulses”. The key transient field-echo (TFE or FLASH) parameters were as follows: number of profiles N=64; TR/TE=6.8/3.5 (ms); FA=13°; shot interval (T_{seg})=2000 (ms); number of shots (or segments)=34; profile order=“low-high”; turbo direction=“radial”; CENTRA (spiral)=“yes”. Each R_{1ρ}-weighted 3D dataset took 1:09 minutes, and a total scan time was 9.2 minutes per Mₚᵣₑₚ.

### Standard R_{1ρ} Mapping Protocol

The acquisition parameters different from those listed above are as follows: ω₁/2π=500 (Hz); TSL=1, 10, 20, 30, 40 (ms); SL method =“rotary-echo” (see FIG. 18A); acquired voxel size=0.4*0.4*3.0 (mm³); TR/TE=12/6.1 (ms); FA=10°, number of shots=52. This protocol took 1:45 minutes to collect each R_{1ρ}-weighted 3D dataset, and a total scan time was 8.75 minutes.

Comparison of R_{1ρ}-Weighted Images with Different FA

One R_{1τ}-weighted scan (TSL=9 ms, ω₁=0) from the developed R_{1ρ} dispersion protocol was repeated with FA of 9°, 11°, 15° and 17° on the first subject's left knee in order to compare with that from an optimum 13°.

### Estimation of Signal-to-Noise Ratio (SNR)

The SNR of the developed R_{1ρ} dispersion imaging was not measured in this study, but it was inferred from the previously acquired five repeated datasets (TSL=1 ms, ω₁/2π=0) using the preliminary R_{1ρ} dispersion protocol based on the standard mapping as aforementioned. The signal mean and SD from each of segmented ROIs in those R_{1ρ}-weighted images were calculated and an average SNR was thus assessed respectively for the femoral, tibial and patellar cartilage compartments.

### In Vivo R_{1ρ} Dispersion Data Analysis

The measured R_{1ρ}-weighted data were fitted to EQUATIONS 8-9 using a free nonlinear curve fitting IDL script based on the Levenberg-Marquardt algorithm (http://purl.com/net/mpfit). Specifically, there were two independent variables (TSL and ω₁) and four model parameters (S0, R₂ⁱ, R₂^{a} and τ_{b}) in this special fitting. The measurement uncertainties for these observed signals were set to unity; accordingly, the output formal 1-sigma fitting errors were scaled so that the reduced chi-squared X² values were approximately equal to one.

The model fit parameters were constrained as follows: S0=[100, 1000]; R₂ⁱ=[1, 20] (1/s); R₂^{a}=[0.5, 100] (1/s) and τ_{b}=[1, 1000] (μs), with initial values set respectively to 500, 10, 20 and 250. If fitted parameters were equal to the boundary values or their relative uncertainties exceeded 100%, these fits would be excluded from further analysis. The goodness of fit was loosely defined by R², indicating to what extent the observed R_{1ρ} dispersion profile could be explained by the fitted model. Paired student's t-tests were used to assess R_{1ρ} differences obtained from between the previous R_{1ρ} mapping methods and the proposed R_{1ρ} dispersion protocol, with significant differences denoted by P<0.05. All measurements are shown as mean±SD unless stated otherwise, and all image and data analysis were conducted with an in-house software developed in IDL 8.5 (Harris Geospatial Solutions, Inc., Broomfield, Colo., USA).

### Results

### An Optimized R_{1ρ} Dispersion Imaging Sequence

Two key components in the SL prepared turbo-FLASH sequence are illustrated in FIGS. 9A-9B. As revealed by Bloch simulations in FIGS. 18A-18D, the proposed SL method (FIGS. 9A and 18D) was more robust to B₀ and B₁ field artifacts with less signal modulation for a wider range of SL strengths (ω₁/2π) particularly when ω₁/2π was relatively weak.

FIG. 9C shows an exemplary R_{1ρ}-weighting map derived from EQUATIONS 8-9 with

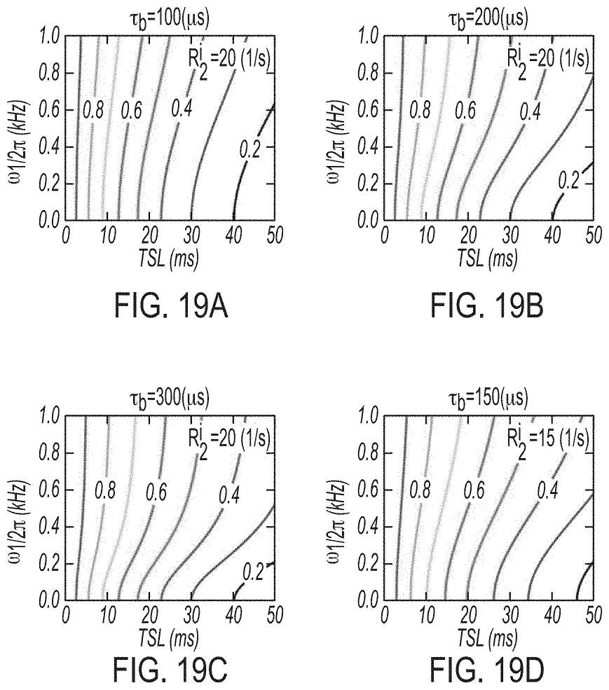

R₂ⁱ=R₂^{a}=20 (1/s), and τ_{b}=300 (μs), where 8 black circles traced an approximately constant Mₚᵣₑₚ of 50% trajectory. The Mₚᵣₑₚ contour plots with τ_{b}=100, 200, 300 (μs) and with τ_{b}=150 (μs) and R₂ⁱ=15 (1/s) are respectively displayed in FIGS. 19A-19D, where the trajectories for Mₚᵣₑₚ of 50% in FIGS. 19C-19D were quite similar.

FIG. 9D demonstrates different Mₚᵣₑₚ evolutions towards steady-state (Mₛₛ) during FLASH imaging readout. For instance, Mₛₛ became around 0.18 with TR/T1=6.8/1240 and FA=13° (see EQUATION 5), and Mₚᵣₑₚ will evolve decreasingly or increasingly when it was larger (green line) or smaller (blue line) than Mₛₛ (red line), respectively. The k-space filling pattern (Ky-Kz, phase-encoding directions) is illustrated in FIG. 9E, where the central region was covered by the first few shots to avoid any potentially involuntary knee movements.

### Simulated Noisy Quantitative R_{1ρ} Dispersion

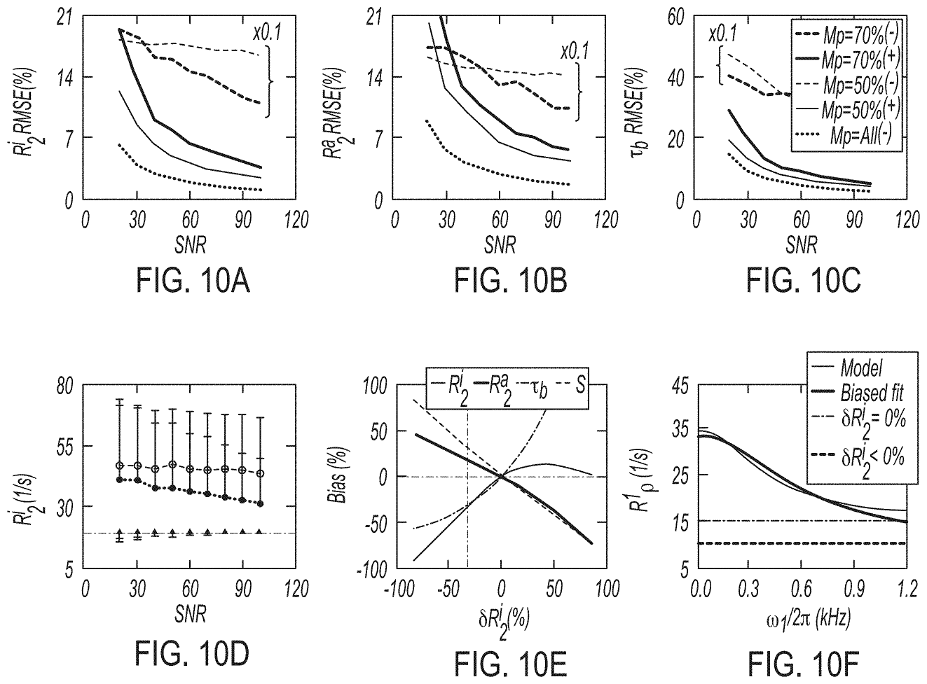

FIGS. 10A-10D show the simulated noisy R_{1ρ} dispersion quantifications with (+, solid line) and without (−, dashed lines) an REF under the influences of varying SNRs. The fitting accuracies, RMSE(%), were significantly improved for R₂ⁱ (FIG. 10A), R₂^{a} (FIG. 10B) and τ_{b} (FIG. 10C) when an REF was included as demonstrated for Mₚᵣₑₚ=50% (blue) and 70% (red). Yet, a slightly decreased RMSE(%) could be attained using a cluster of Mₚᵣₑₚ (i.e. All=50%+60%+70%, black) without an REF. FIG. 10D proves that a relatively precise R₂ⁱ could be realized using either one Mₚᵣₑₚ with an REF or a cluster of Mₚᵣₑₚ without an REF. TABLE 4 lists the mean (n=1000) and SD of each fitted parameter with SNR=30, 60 and 90. In general, the quantification accuracy of R_{1ρ} dispersion improves progressively or dramatically when SNR increases or comprising an REF, respectively. It is worth indicating that these simulated results did not consider any potential biases stemmed from the related imaging readout.

If an REF had not been reliably identified in reality, the expected (red sold line) R_{1ρ} dispersion characterization would have been compromised (black solid line) as revealed in FIG. 10F, where R₂ⁱ was reduced intentionally from its input value of 15 (1/s) (red dashed line) to a biased 10 (1/s) (black dashed line) when modeling an erroneous REF. As a result, the fitted parameters would become biased as indicated in FIG. 10E (vertical dashed line), where a range of relative uncertainties δR₂ⁱ, from −100% to 100%, were considered. Largely, δR₂ⁱ is proportional to δτ_{b}(green) but inversely proportional to δR₂ⁱ (red) and δS (blue).

### An Optimal FA and Estimated SNRs

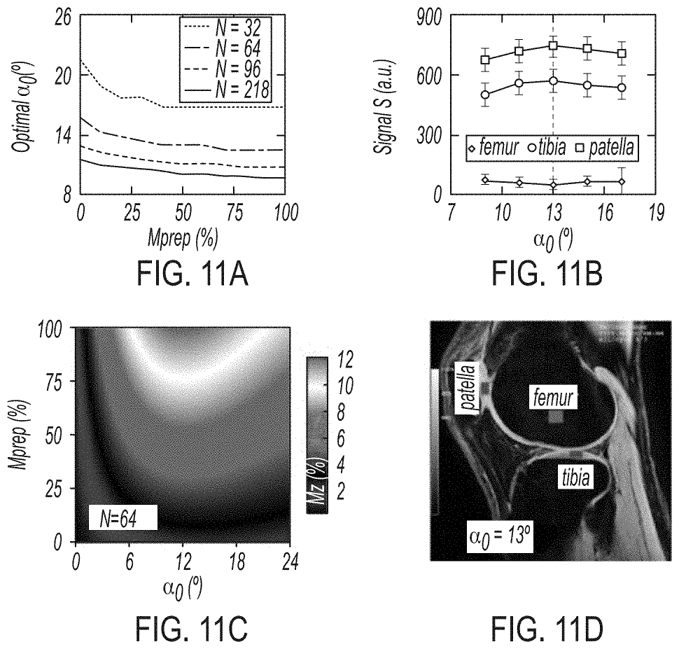

For different N and initial Mₚᵣₑₚ, an optimal FA could be calculated (FIG. 11B) with TR/T₁=6.8 1240 (EQUATIONS 5-7). For instance, these optima decreased gradually from 15.6° to 12.3° (N=64, green line) when Mₚᵣₑₚ increased from 0 and 100% (FIG. 11A); therefore, an optimal FA of 13° was used in this study. Nonetheless, these predicted optima would have been respectively decreased or increased if N had been increased or decreased, consistent with a previous finding. Compared with other FAs (FIG. 11C), an ROI-based R_{1ρ}-weighted signal became the largest when using FA=13° in the tibial (green circle) or patellar (blue square) cartilage (FIG. 11D). However, this was not the case for the signals from the femoral condyle (red diamond) and muscle near the knee (data not shown). Quantitatively, a signal increase of less than 10% was found when FA changed from a previously used 10° to an optimum 13°.

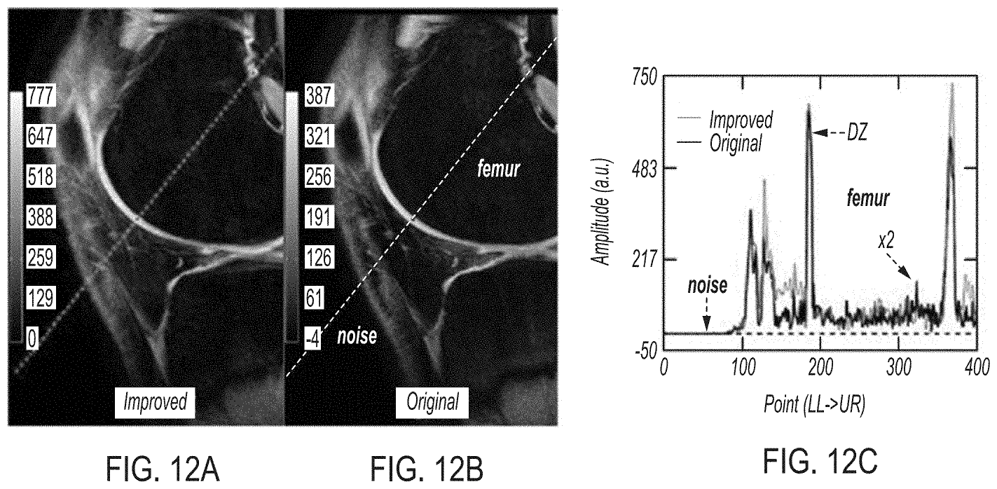

The SNR of R_{1ρ}-weighted image was estimated using previously acquired datasets (n=5); specifically, the femoral, tibial and patellar cartilage had respectively SNR of 66.5±13.6, 107.0±23.5 and 69.3±13.9. Although some original acquisition parameters (e.g. FA, voxel size and SL scheme) had been altered, the developed (i.e. improved) R_{1ρ} dispersion imaging protocol could still generate a comparable SNR, as demonstrated by two overlaid line profiles (FIG. 12C) taken from the same anatomical location (FIG. 12A-12B). It is worth noting that both the improved (TSL=21 ms, FIG. 12A) and the original (TSL=20 ms, FIG. 12B) R_{1ρ}-weighted images were acquired using ω₁/2π=500 (Hz), and that the total scan time was only 1:09 minutes for the former and 1:45 minutes for the latter.

### In Vivo Quantitative R_{1ρ} Dispersion Imaging

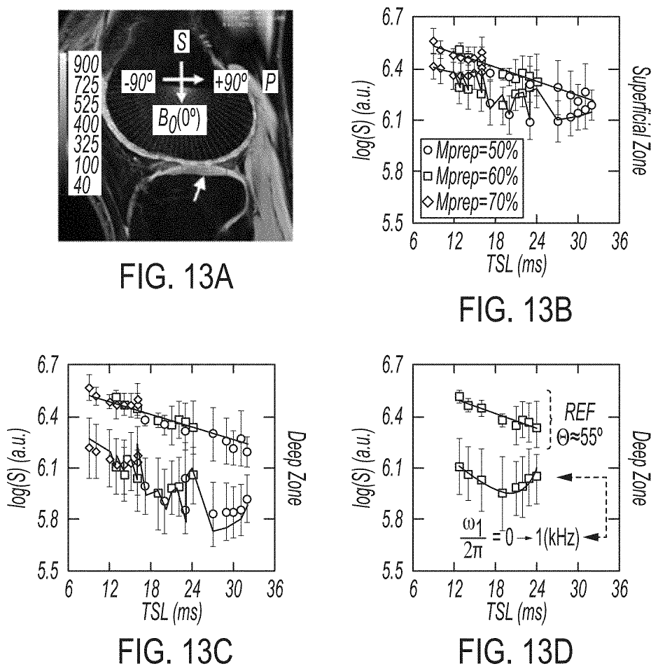

FIG. 13 illustrates some exemplary measured (colored symbols) and modeled (black lines) cartilage R_{1ρ} dispersion profiles from the left knee of the first subject. These examples were obtained from two radially-segmented ROIs in the superficial (FIG. 13B) and the deep (FIG. 13C) tibial cartilage (FIG. 13A, yellow arrow). Here, all measurements with Mₚᵣₑₚ=50% (red), 60% (green) and 70% (blue) were considered together, and the R_{1ρ} dispersion fitting incorporated an REF1 measured in the deep femoral cartilage. FIG. 13D replots the data of Mₚᵣₑₚ=60% from FIG. 13C, with a straight line (black) standing for the fit of an REF1 data (i.e. equivalent to ω₁=∞) and a curved line (black) for the fit of the measured R_{1ρ} dispersion profile using ω₁/2π from 0 to 1 kHz (see TABLE 3) and TSL from 13 to 24 (ms).

It was clear that R_{1ρ} became significantly (P<0.01) less dispersed in the superficial zone (SZ) than in the deep zone (DZ), with the least at the MA orientation; specifically, the fitted R₂^{a}(1/s), τ_{b} (μs) and S (10⁻³) were respectably 14.8±0.9 vs. 27.6±1.3, 205±17 vs. 104±8 and 2.13±0.11 vs. 4.07±0.19 in the SZ and DZ. Further analyses for each group were also performed and the fitted R₂ⁱ, R₂^{a}, S and τ_{b}, with (+) and without (−) an REF1, are tabulated in TABLE 5.

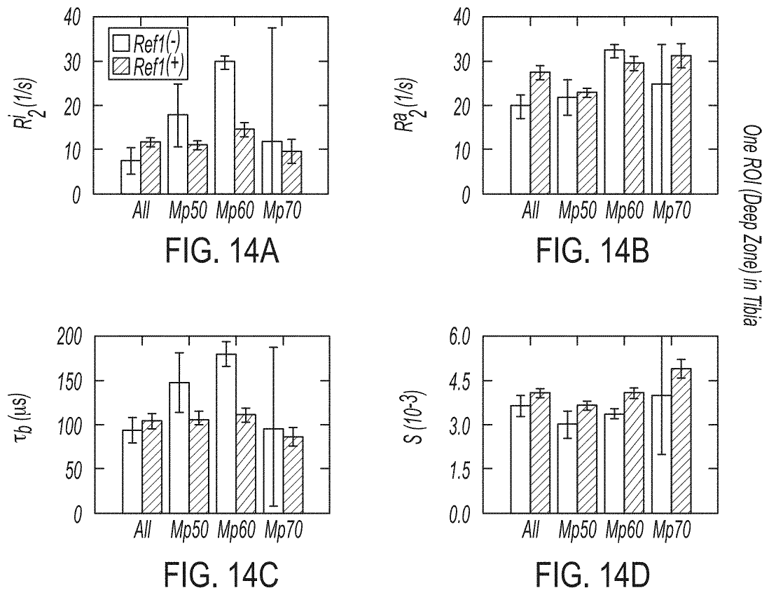

FIG. 14 compares the resulting fits in the DZ, showing that the precisions of the fits (i.e. error bars) were markedly improved as predicted by the previous simulations (FIG. 2) when including an REF1 (red bars). Taken the “All” Mₚᵣₑₚ group without an REF1 (blue bars) as a reference, a single group of Mₚᵣₑₚ=50% or 60% with an REF1 generated a relatively more accurate quantification than that from Mₚᵣₑₚ=70%. This observation was possibly accounted for by an unreliable REF1 derived from a relatively limited TSL range used for Mₚᵣₑₚ=70% (see TABLE 3)

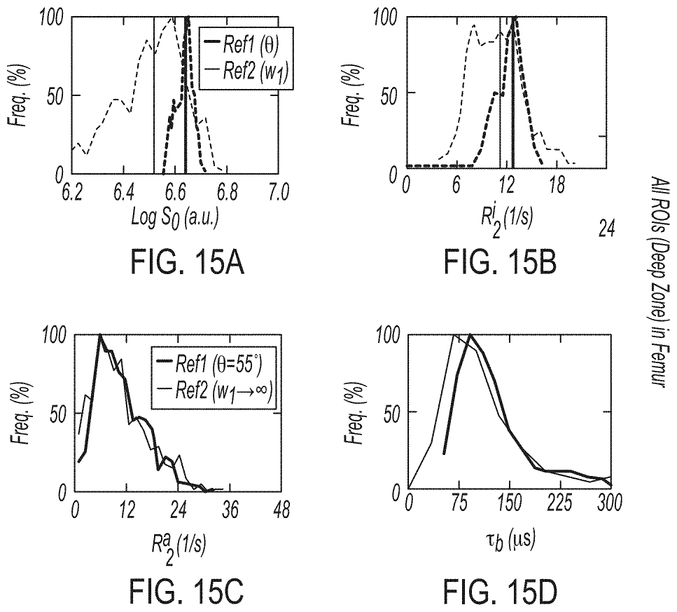

As revealed in FIGS. 15A-15B, when all segmented ROIs in the deep femoral cartilage were considered, an average fitted S₀ in logarithmic scale (FIG. 15A) or R₂ⁱ (FIG. 15B) from the “All” Mₚᵣₑₚ group without an REF1 (blue solid line) was very close to that from Mₚᵣₑₚ=60% with an REF1 (red solid line), i.e. 6.64±0.03 vs. 6.52±0.13 and 12.8±1.7 vs. 11.3±3.0 (1/s). These average S₀ and R₂ⁱ from the “All” group could be used to generate an REF2 corresponding to ω₁=∞. When including either an REF1 (red) or an REF2 (blue) in quantifying the observed R_{1ρ} dispersion profiles, no statistically significant differences could be found between the fitted R₂^{a}(1/s) as shown in FIG. 15C (i.e. 11.6±5.9 vs. 11.5±6.7, P=0.95) and the fitted τ_{b} (μs) in FIG. 15D (i.e. 139±64 vs. 134±80, P=0.85). This in vivo observation is in consistent with the presented cartilage R_{1ρ} dispersion theory (EQUATION 9).

### An Orientation-Independent Order Parameter S

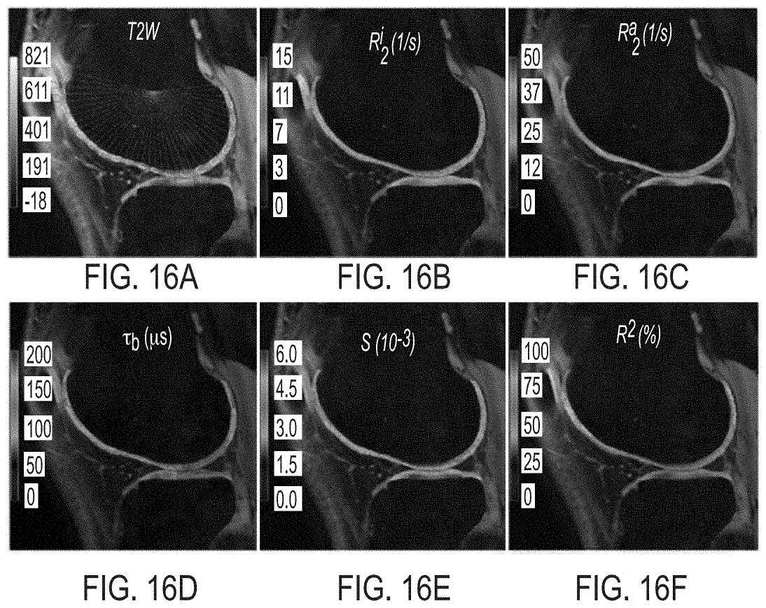

An exemplary quantitative cartilage R_{1ρ} dispersion (Mₚᵣₑₚ=60%) of the third subject's left knee is presented in FIG. 16. Particularly, an anatomical T2W sagittal image was shown in FIG. 16A superimposed with angularly and radially segmented ROIs, and the ROI-based parametric maps (R₂ⁱ, R₂^{a}, τ_{b}, S and R²) were respectively overlaid upon the T2W image in FIGS. 16B-16F. Less reliable quantification was evident particularly around the trochlear cartilage as indicated by reduced R² values (FIG. 16F), resulting from a vanishing residual dipolar coupling near the MA orientation.

With respect to the fitted R₂^{a} and τ_{b} (FIGS. 16C-16D), the orientation dependences of the fitted R₂ⁱ (FIG. 16B) and S (FIG. 16E) were markedly reduced, which was not unexpected. In a longitudinal cartilage study, this derived order parameter S should remain unchanged for a specific location unless collagen is somewhat depleted due to OA. A summary of quantitative R_{1ρ} dispersion (Mₚᵣₑₚ=60%) for all knees including repeated scans is provided in TABLE 6, with the fitted parameters derived from segmented ROIs in the DZ and SZ of femoral, tibial and patellar cartilage. It is worth mentioning that it was a challenging task to manually segment the DZ from the calcified cartilag. Thus, it would not be surprised to observe some abrupt changes of R₂^{a} in the DZ such as in the tibial cartilage (FIG. 16C).

Synthetic and Measured R_{1ρ} with ω₁/2π=500 Hz

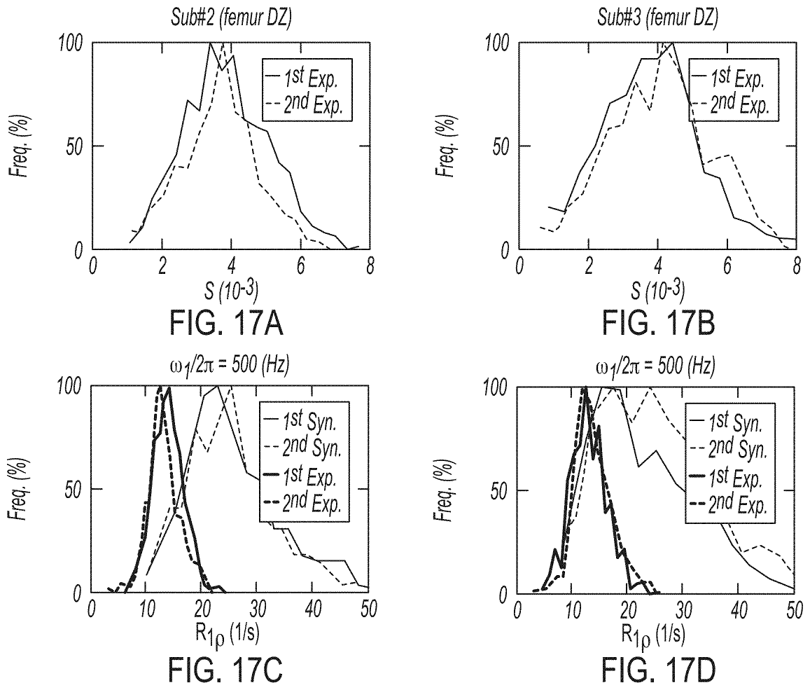

Considering the femoral DZ only from the second (FIGS. 17A and 17C) and the third subject (FIGS. 17B and 17D), the developed R_{1ρ} dispersion imaging protocol (blue) exhibited good reproducibility based on two repeated scans (solid and dashed). For example, the derived average order parameters S (10⁻³) were 4.03±1.21 vs. 3.82±1.14 (p =.57, FIG. 17A) and 4.09±1.54 vs. 4.28±1.4 (p=0.69, FIG. 17B), respectively.

A measured (red) and a synthetic (blue) R_{1ρ} distribution are compared in FIGS. 17C-17D, showing that an average measured value (1/s) was significantly smaller than that of the synthetic, e.g. 13.8±3.0 vs. 26.9±8.3, p<10⁻⁴, for the second scan from the second subject (solid, FIG. 17C). These findings agree well with a recent multi-vendor and multi-site R_{1ρ} quantification of knee cartilage study, indicating that R_{1ρ} was greatly underestimated using a conventional fast gradient-echo sequence.

Furthermore, the overall synthetic R_{1ρ} from these two subjects, as tabulated in TABLE 7, was not significantly (p=0.71) different from that measured by the state-of-the-art 3D MAPSS sequence, i.e. 24.4±6.0 vs. 23.6±2.9 (1/s), suggesting that the developed R_{1ρ} dispersion imaging protocol was also less sensitive to the transient magnetization evolution artifacts. These reported R_{1ρ} relaxation rates would have become 41.0±10.2 vs. 42.4±5.2 (ms) if they had been expressed with T_{1ρ} relaxation time constants (i.e. T_{1ρ}=1/R_{1ρ}).

### Discussion

This work presents an efficient and robust R_{1ρ} dispersion imaging protocol that can provide a unique MR imaging biomarker specifically related to collagen changes in highly ordered tissues such as human knee articular cartilage in clinical studies. This new method was developed based on previous findings including R_{1ρ} relaxation dispersion mechanism, and corroborated by in vivo knee imaging and simulation studies. The comparison results suggest that much more detailed R_{1ρ} dispersion characterization could be attained within a similar scan duration normally used for the conventional R_{1ρ} mapping.

### Restricted Water Molecular Reorientation Correlation Time τ_{b}

Although a plethora of in vivo knee cartilage R_{1ρ} mapping research has been performed in the past, only two quantitative R_{1ρ} dispersion studies can be found in the literature. The functional form of R_{1ρ} dispersion turned out to be a kind of Lorentzian function regardless of the reported relaxation mechanisms. The so-called inflection point (ωᵢₚ) on R_{1ρ} dispersion profile could be determined by setting the second derivative of such a Lorentzian function to zero, which is directly linked to the characteristically slow molecular motion time scale, i.e. 1/τ_{b}=2\sqrt{3}*ωᵢₚ based on EQUATION 9. The measured ωᵢₚ values on in vivo human knee cartilage at 3T have been reported previously, and an average τ_{b} (μs) was calculated as 262±58, with a minimum and maximum of 168 and 420, respectively. These rough estimates are in good agreement with previous findings. Therefore, it was not unreasonable to select τ_{b} of 300 μs for numerical simulations and for determining the tailored TSL and ω₁ values as listed in TABLE 3.

### An Optimal FA for FLASH Sequence

An empirical relationship between an optimal FA, θₒₚₜ(°), and the number of profiles, N, was given as θₒₚₜ=\sqrt{8192/N}, assuming that Mₚᵣₑₚ was 100% and an effect of longitudinal T₁ relaxation was negligible (i.e. T₁=∞) during FLASH imaging readout. In the case of a finite τ₁=1240 ms for cartilage and TR=6.8 ms, an optimal FA should become relatively larger to compensate for some magnetization loss due to the finite T₁ relaxation.

For instance, an optimal FA (N=64, Mₚᵣₑₚ=100%.) would become 12.3° and 11.3°, respectively, with and without considering T₁ relaxation. Nonetheless, an approximately quadratic decrease in θₒₚₜ could still be observed when N progressively increased from 32 to 128 as shown in FIG. 11A.

### An Efficient Quantitative R_{1ρ} Dispersion Protocol

Even though the acquisition time was reduced by about 30% (1:09 vs. 1:45 minutes) for one R_{1ρ}-weighted dataset when using the developed R_{1ρ} dispersion rather than the previous standard R_{1ρ} mapping protocol, a comparable SNR as demonstrated in FIG. 12 could still be attained. This result could be largely attributed to a larger pixel size (i.e. 0.6*0.6 vs. 0.4*0.4 mm²) and a fully-refocused SL preparation being used in the proposed method. Although 8 R_{1ρ}-weighting 3D images were acquired per Mₚᵣₑₚ in this study and incorporated an internal reference (i.e. 8 extra data points) to fit four model parameters, only two acquisitions would suffice. In fact, this unique concept has been employed in previous work to derive an anisotropic R₂^{a} from a single τ₂-weighed image.

There still exists ample room for further improvement of the developed R_{1ρ} dispersion imaging protocol; for instance, a dramatic change on knee cartilage R_{1ρ} dispersion profile should occur around ωᵢₚ/2π=200 Hz as reported, and thus the ω₁ distribution should have been tailored accordingly to maximize the sensitivity of R_{1ρ} dispersion imaging. Moreover, the reported ω₁ ranges need to be modified if MR scanner hardwire does not afford the highest SL RF strength of 1000 Hz. In this work, a dedicated 16-channel transmit/receive knee coil was employed that could generate a maximum B₁ of about 27 μT, equivalent to ω₁/2π=1150 Hz on the 3T MR scanner.

### Dispersed and Non-Dispersed R_{1ρ} Components

The theoretical basis for the developed R_{1ρ} dispersion imaging protocol relies on the fact that R_{1ρ} relaxation can be accounted for by two leading contributions, i.e. the non-dispersed and dispersed parts. In the case of articular cartilage as shown by EQUATION 9, these two contributions are an isotropic R₂ⁱ and an anisotropic R₂^{a}, assuming a negligible chemicall exchange R₂^{ex}. This biophysical understanding of R_{1ρ} dispersion mechanism is fully aligned with an insightful view from the literature in that small amount of water molecules hidden within the triple-helix interstices in collagen microstructure becomes mainly responsible for the observed R_{1ρ} dispersion.

Such an insight into R_{1ρ} relaxation mechanism not only warrants the specificity of the derived MR relaxation metrics such as R₂^{a} and S, but also provides an opportunity to exploit other valuable information without any additional scan time. In the previous and the current work, an internal reference was used to facilitate R_{1ρ} dispersion modeling. In an ideal scenario as shown in EQUATION 9, this reference information represented by R₂ⁱ should be the same whether it is determined when θ=55° (REF1) or when ω₁=∞ (REF2). Nevertheless, if R₂^{ex} is included at the magic angle orientation (i.e. R₂^{a}=0) even it is insignificant in other cartilage locations (i.e. R₂^{a}>>R₂^{ex}), REF2 (i.e. R₂ⁱ) would be less than REF1 (i.e. R₂ⁱ+R₂^{ex}) just as appeared in FIG. 15B. It is quite likely that the observed difference between REF1 and REF2 could have been larger if REF1 had not been underestimated due to the specific femoral condyle geometry. This was because that some deep femoral cartilage in sagittal imaging slices had not been adequately characterized by a function of R₂^{a} [symbol] 3 cos² θ−1 [symbol] ²/4.

Measuring an Unbiased R_{1ρ} with FLASH Sequence

The primary utility of 3D MAPSS was to measure an accurate R_{1ρ} of human knee cartilage by eliminating an adverse longitudinal relaxation effect, which was manifested by a varying k-space filtering for different prepared magnetizations. Without such a dedicated attention, R_{1ρ} could be markedly underestimated as demonstrated in a recent multi-center and multi-vendor knee cartilage R_{1ρ} mapping study. Similarly, the current study also confirmed the previous findings as shown in FIGS. 17C and 17C in which the observed R_{1ρ} was greatly reduced when using the standard R_{1ρ} mapping.

On the other hand, the overall synthetic R_{1ρ} (ω₁/2π=500 Hz) values from this study are comparable with that measured with 3D MAPPS, suggesting that the developed R_{1ρ} dispersion imaging method is not only efficient but also robust—free from the T₁ relaxation effect during FLASH imaging readout. Recently, an efficient 3D MAPSS without RF phase cycling was reported for a robust neuro R_{1ρ} mapping using a different variable flip-angle scheduling tailored to various prepared R_{1ρ} magnetization. This improved 3D MAPSS method would be cumbersome if it is used for R_{1ρ} dispersion imaging, and the SL preparation has not yet been optimized. As demonstrated in FIG. 16, much more information could be derived from the proposed efficient method; for instance, a standard R_{1ρ} mapping (i.e. R₂ⁱ+R₂^{a}), synthetic R_{1ρ} mapping with any ω₁/2π value. Most importantly, an orientation-independent order parameter S can be determined for both longitudinal and cross-sectional clinical studies of human knee articular cartilage.

### Conclusions

An efficient and robust R_{1ρ} dispersion imaging protocol that is less susceptible to imaging artifacts from non-uniform B₀ and B₁ fields during SL preparation and from an adverse T₁ relaxation effect during FLASH imaging readout has been developed. While the proposed method was developed and demonstrated on human knee articular cartilage, its application may be expanded to other biological tissues and relevant disorders, such as liver fibrosis and intervertebral disc degeneration, already being studied by standard R_{1ρ} mapping. Continued refinement of R_{1ρ} relaxation dispersion methodology will facilitate additional insight into pathophysiological processes, more accurate diagnoses, and better characterization of treatment efficacy in clinical joint cartilage studies.

### Exemplary System

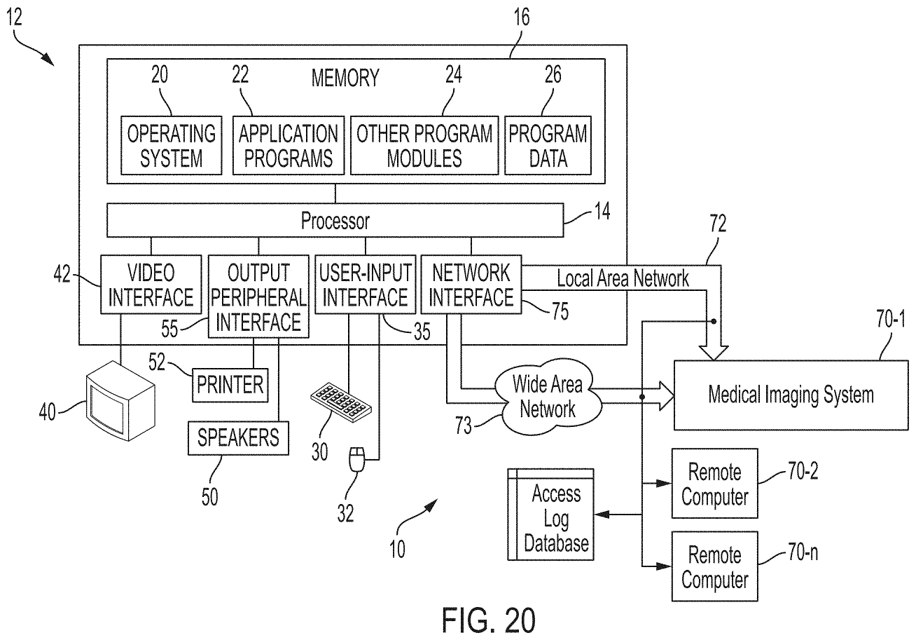

With reference to FIG. 20, an exemplary system for implementing the blocks of the method and apparatus includes a general-purpose computing device in the form of a computer 12. Components of computer 12 may include, but are not limited to, a processing unit 14 and a system memory 16. The computer 12 may operate in a networked environment using logical connections to one or more remote computers, such as remote computers 70-1, 70-2, . . . 70-n, via a local area network (LAN) 72 and/or a wide area network (WAN) 73 via a modem or other network interface 75. These remote computers 70 may include other computers like computer 12, but in some examples, these remote computers 70 include one or more of (i) a medical imaging system, such as magnetic resonance imaging (MRI) device, (ii) a signal records database systems, (iii) a scanner, and/or (v) a signal filtering system.

In the illustrated example, the computer 12 is connected to a medical imaging system 70-1. The medical imaging system 70-1 may be a stand-alone system capable of performing imaging of molecules, such as water, in biological tissue for in vivo examination. The system 70-1 may have resolution of such biological features as fibers, membranes, micromolecules, etc., wherein the image data can reveal microscopic details about biological tissue architecture, in a normal state or diseased state.

Computer 12 typically includes a variety of computer readable media that may be any available media that may be accessed by computer 12 and includes both volatile and nonvolatile media, removable and non-removable media. The system memory 16 includes computer storage media in the form of volatile and/or nonvolatile memory such as read only memory (ROM) and random access memory (RAM). The ROM may include a basic input/output system (BIOS). RAM typically contains data and/or program modules that include operating system 20, application programs 22, other program modules 24, and program data 26. The computer 12 may also include other removable/non-removable, volatile/nonvolatile computer storage media such as a hard disk drive, a magnetic disk drive that reads from or writes to a magnetic disk, and an optical disk drive that reads from or writes to an optical disk.

A user may enter commands and information into the computer 12 through input devices such as a keyboard 30 and pointing device 32, commonly referred to as a mouse, trackball or touch pad. Other input devices (not illustrated) may include a microphone, joystick, game pad, satellite dish, scanner, or the like. These and other input devices are often connected to the processing unit 14 through a user input interface 35 that is coupled to a system bus, but may be connected by other interface and bus structures, such as a parallel port, game port or a universal serial bus (USB). A monitor 40 or other type of display device may also be connected to the processor 14 via an interface, such as a video interface 42. In addition to the monitor, computers may also include other peripheral output devices such as speakers 50 and printer 52, which may be connected through an output peripheral interface 55.

### Exemplary Method

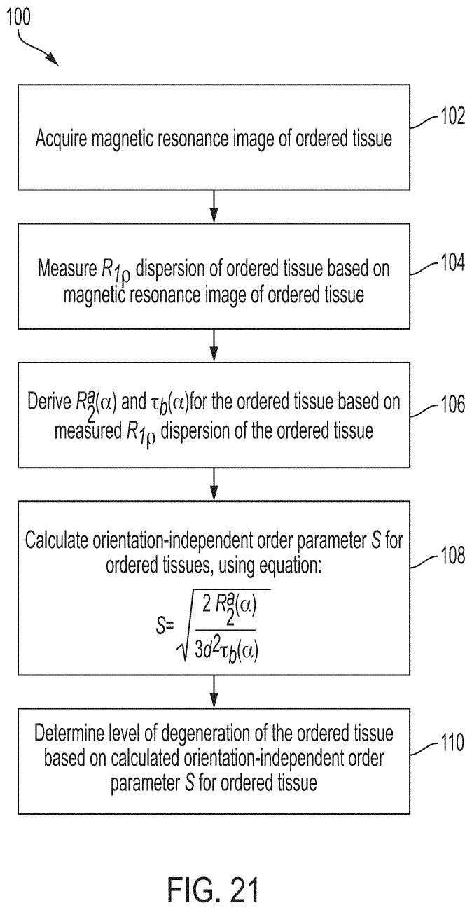

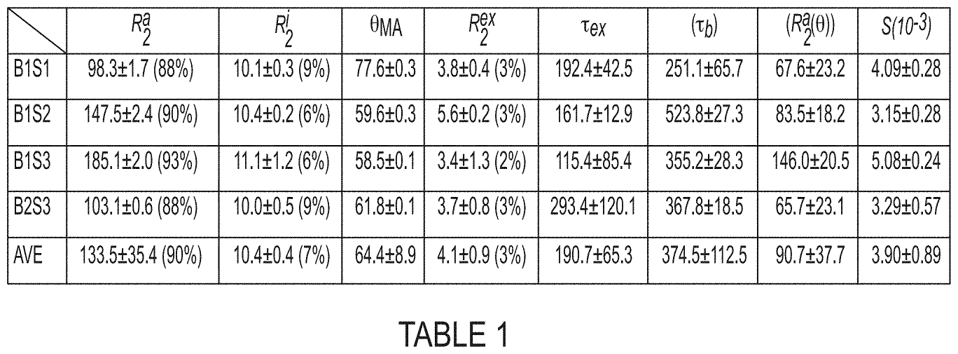

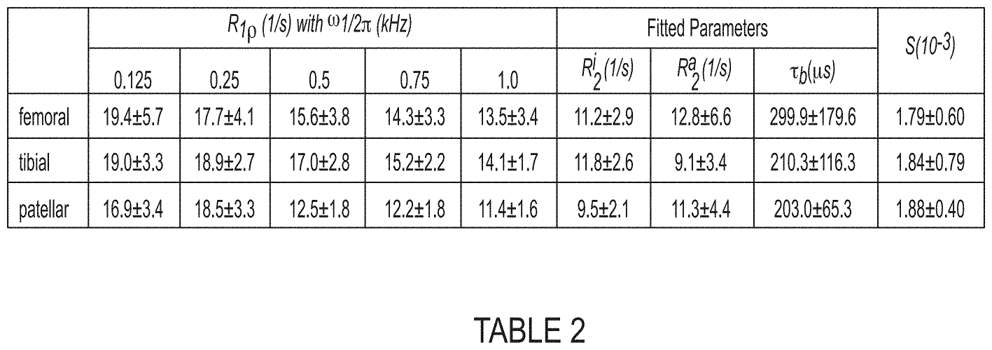

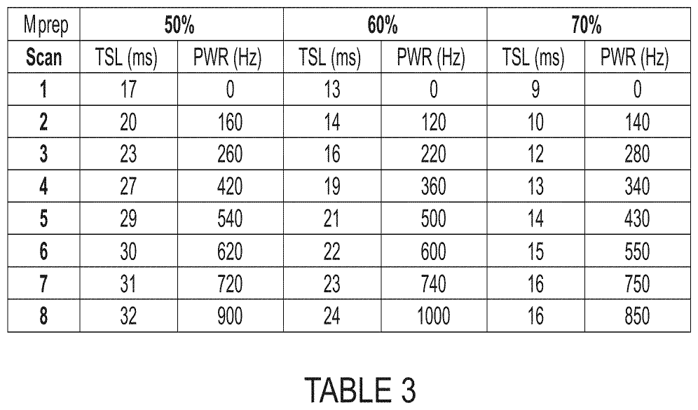

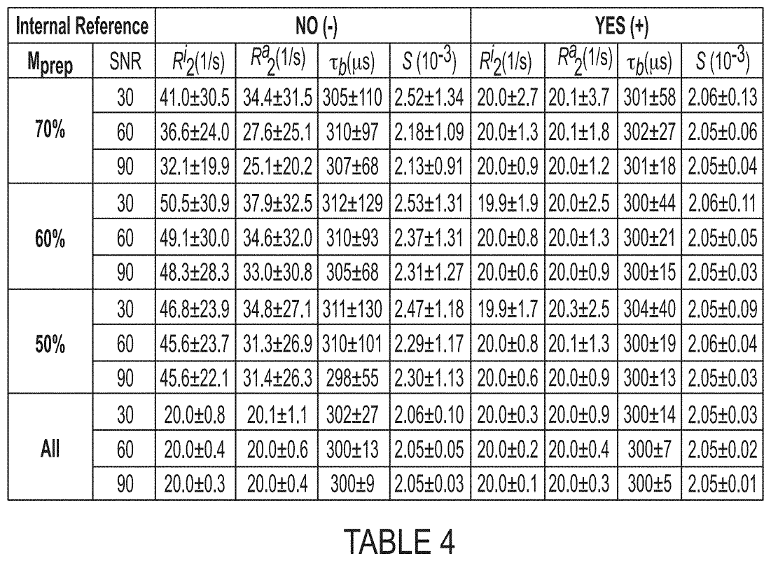

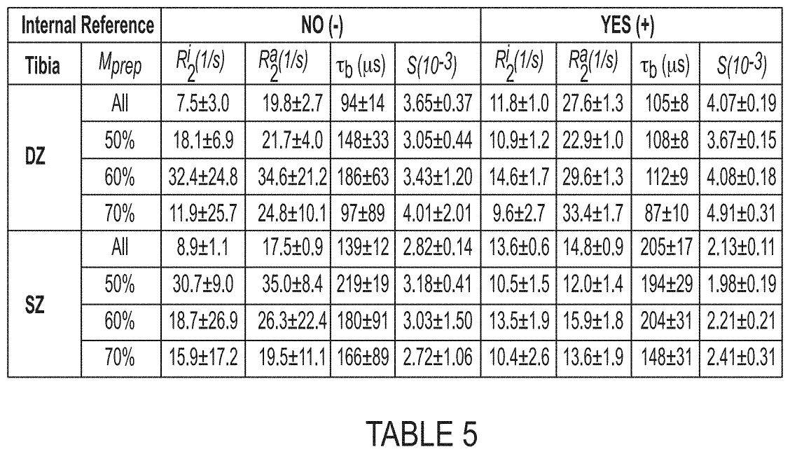

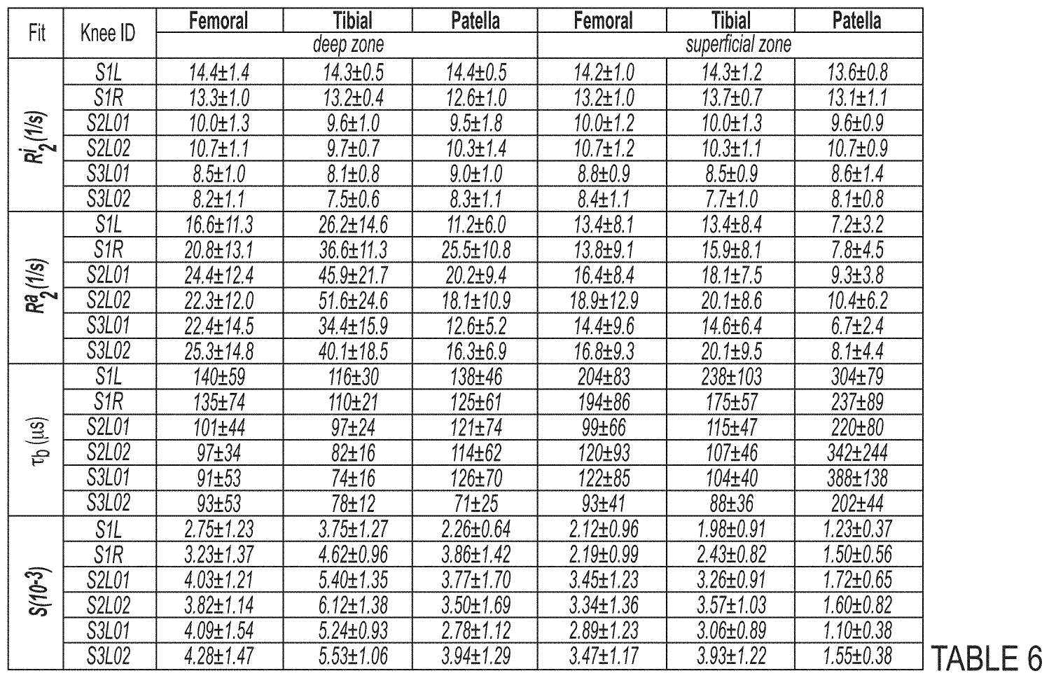

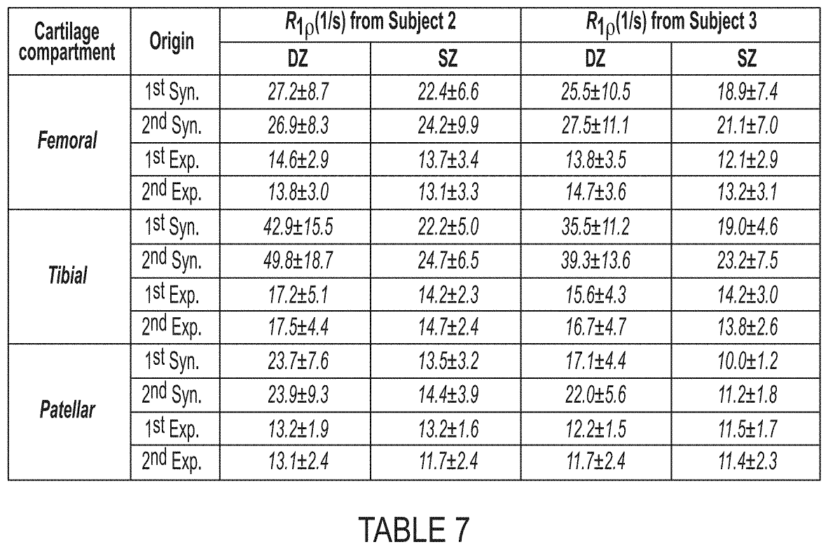

Referring now to FIG. 21, a flow diagram of an exemplary method 100 of analyzing ordered tissue to calculate an orientation-independent order parameter S that is sensitive to the microstructural integrity of cartilage is illustrated in accordance with an embodiment. The method 100 can be implemented as a set of instructions stored on a computer-readable memory and executable on one or more processors.

A magnetic resonance image of an ordered tissue may be acquired (block 102). For example, the ordered tissue may be nerve tissue, white matter tissue, intervertebral disk, skeletal muscle tissue, myocardial muscle tissue, tendon tissue, cartilage tissue, or any other highly structured or highly ordered tissue in the human body.

Based on the magnetic resonance image of the ordered tissue, an R_{1ρ} dispersion of the ordered tissue may be measured (block 104). Based on the measured R_{1ρ} dispersion of the ordered tissue, R₂^{a}(α) and τ_{b}(α) for the ordered tissue may be derived (block 106).

An orientation-independent order parameter S for the ordered tissue may be calculated (block 108) using the following equation:

$$S = \sqrt{\frac{2}{3 {d}^{2}} \frac{{R}_{2}^{a} ( α )}{{τ}_{b} ( α )}} .$$

For example, a lower value for the orientation-independent order parameter S may correspond to a greater degeneration of the ordered tissue, while a higher value for the orientation-independent order parameter S may correspond to a lesser degeneration of the ordered tissue.

Based on the orientation-independent order parameter S for the ordered tissue, a level of degeneration of the ordered tissue may be determined (block 110). Moreover, in some examples, an indication of osteoarthritis in a patient associated with the ordered tissue may be determined based on the orientation-independent order parameter S for the ordered tissue. For instance, an orientation-independent order parameter S for the ordered tissue below a certain threshold value may indicate that the patient associated with the ordered tissue likely suffers from osteoarthritis.

### Additional Considerations

Although the preceding text sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

Throughout this specification, unless indicated otherwise, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may likewise be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, in some embodiments, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” or “in some embodiments” in various places in the specification are not necessarily all referring to the same embodiment or embodiments.

Some embodiments may be described using the terms “coupled,” “connected,” “communicatively connected,” or “communicatively coupled,” along with their derivatives. These terms may refer to a direct physical connection or to an indirect (physical or communication) connection. For example, some embodiments may be described using the term “coupled” to indicate that two or more elements are in direct physical or electrical contact. The term “coupled,” however, may also mean that two or more elements are not in direct contact with each other, but yet still co-operate or interact with each other. Unless expressly stated or required by the context of their use, the embodiments are not limited to direct connection.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless the context clearly indicates otherwise.

Upon reading this disclosure, those of skill in the art will appreciate still additional alternative structural and functional designs for monitoring refrigerated air usage. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

Finally, the patent claims at the end of this patent application are not intended to be construed under 35 U.S.C. § 112(f), unless traditional means-plus-function language is expressly recited, such as “means for” or “step for” language being explicitly recited in the claims.

What is claimed is:

1. A computer-implemented method, comprising:
acquiring, by a processor, a magnetic resonance image of an ordered tissue;
measuring, by a processor, based on the magnetic resonance image of the ordered tissue, an R_{1ρ} dispersion of the ordered tissue;
deriving, by a processor, R₂^{a}(α) and τ_{b}(α) for the ordered tissue based on the measured R_{1ρ} dispersion of the ordered tissue;
calculating, by a processor, an orientation-independent order parameter S for the ordered tissue, using the following equation:
$$S = \sqrt{\frac{2}{3 {d}^{2}} \frac{{R}_{2}^{a} ( α )}{{τ}_{b} ( α )}} ;$$
and
determining, by a processor, based on the orientation-independent order parameter S for the ordered tissue, a level of degeneration of the ordered tissue.

2. The computer-implemented method of claim 1, wherein a lower value for the orientation-independent order parameter S corresponds to a greater degeneration of the ordered tissue, and wherein a higher value for the orientation-independent order parameter S corresponds to a lesser degeneration of the ordered tissue.

3. The computer-implemented method of claim 1, further comprising:
determining, by a processor, an indication of osteoarthritis in a patient associated with the ordered tissue based on the orientation-independent order parameter S for the ordered tissue.

4. The computer-implemented method of claim 3, wherein determining an indication of osteoarthritis in a patient associated with the ordered tissue based on the orientation-independent order parameter S for the ordered tissue comprises:
determining an indication of osteoarthritis in a patient associated with the ordered tissue based on the orientation-independent order parameter S for the ordered tissue being below a certain threshold value.

5. The computer-implemented method of claim 1, wherein the ordered tissue is one of: nerve tissue, white matter tissue, intervertebral disk, skeletal muscle tissue, myocardial muscle tissue, tendon tissue, or cartilage tissue.

6. A system, comprising:
a magnetic resonance imaging (MRI) device configured to capture a magnetic resonance image of an ordered tissue;
one or more processors; and
one or more memories storing instructions that, when executed by the one or more processors, cause the one or more processors to:
measure, based on the magnetic resonance image of the ordered tissue, an R_{1ρ} dispersion of the ordered tissue;
derive R₂^{a}(α) and τ_{b}(α) for the ordered tissue based on the measured R_{1ρ} dispersion of the ordered tissue;
calculate an orientation-independent order parameter S for the ordered tissue, using the following equation:
$$S = \sqrt{\frac{2}{3 {d}^{2}} \frac{{R}_{2}^{a} ( α )}{{τ}_{b} ( α )}} ;$$
and
determine, based on the orientation-independent order parameter S for the ordered tissue, a level of degeneration of the ordered tissue.

7. The system of claim 6, wherein a lower value for the orientation-independent order parameter S corresponds to a greater degeneration of the ordered tissue, and wherein a higher value for the orientation-independent order parameter S corresponds to a lesser degeneration of the ordered tissue.

8. The system of claim 6, wherein the instructions further cause the processors to:
determine an indication of osteoarthritis in a patient associated with the ordered tissue based on the orientation-independent order parameter S for the ordered tissue.

9. The system of claim 8, wherein determining an indication of osteoarthritis in a patient associated with the ordered tissue based on the orientation-independent order parameter S for the ordered tissue comprises:
determining an indication of osteoarthritis in a patient associated with the ordered tissue based on the orientation-independent order parameter S for the ordered tissue being below a certain threshold value.

10. The system of claim 6, wherein the ordered tissue is one of: nerve tissue, white matter tissue, intervertebral disk, skeletal muscle tissue, myocardial muscle tissue, tendon tissue, or cartilage tissue.

11. A tangible, non-transitory computer-readable medium storing executable instructions that when executed by at least one processor of a computing device, cause the computing device to:
acquire a magnetic resonance image of an ordered tissue;
measure, based on the magnetic resonance image of the ordered tissue, an R_{1ρ} dispersion of the ordered tissue;
derive R₂^{a}(α) and τ_{b}(α) for the ordered tissue based on the measured R_{1ρ} dispersion of the ordered tissue;
calculate an orientation-independent order parameter S for the ordered tissue, using the following equation:
$$S = \sqrt{\frac{2}{3 {d}^{2}} \frac{{R}_{2}^{a} ( α )}{{τ}_{b} ( α )}} ;$$
and
determine, based on the orientation-independent order parameter S for the ordered tissue, a level of degeneration of the ordered tissue.

12. The tangible, non-transitory computer-readable medium of claim 11, wherein a lower value for the orientation-independent order parameter S corresponds to a greater degeneration of the ordered tissue, and wherein a higher value for the orientation-independent order parameter S corresponds to a lesser degeneration of the ordered tissue.

13. The tangible, non-transitory computer-readable medium of claim 11, wherein the instructions further cause the computing device to:
determine an indication of osteoarthritis in a patient associated with the ordered tissue based on the orientation-independent order parameter S for the ordered tissue.

14. The tangible, non-transitory computer-readable medium of claim 13, wherein determining an indication of osteoarthritis in a patient associated with the ordered tissue based on the orientation-independent order parameter S for the ordered tissue comprises:
determining an indication of osteoarthritis in a patient associated with the ordered tissue based on the orientation-independent order parameter S for the ordered tissue being below a certain threshold value.

15. The tangible, non-transitory computer-readable medium of claim 11, wherein the ordered tissue is one of: nerve tissue, white matter tissue, intervertebral disk, skeletal muscle tissue, myocardial muscle tissue, tendon tissue, or cartilage tissue.
